(19) United States
(12) Patent Application Publication
(10) Pub. No.: US 2009/0214552 A1
(43) Pub. Date: Aug. 27, 2009

(54) Novel Egg Receptors for Sperm Proteins

(75) Inventors: Arabinda Mandal, Charlottesville, VA (US); Monika Sachdev, Charlottesville, VA (US); John C. Herr, Charlottesville, VA (US)

Correspondence Address: UNIVERSITY OF VIRGINIA PATENT FOUNDATION, 250 WEST MAIN STREET, SUITE 300, CHARLOTTESVILLE, VA, 22902, US

(73) Assignee: UNIVERSITY OF VIRGINIA PATENT FOUNDATION, Charlottesville, VA (US)

(21) Appl. No.: 11/915,225

(22) PCT Filed: Feb. 21, 2006
(86) PCT No.: PCT/US2006/005970

§ 371 Date: Nov. 24, 2008

Related U.S. Application Data
(60) Provisional application No. 60/655,562, filed on Feb. 23, 2005.

(51) Int. Cl. A61K 39/395; A61K 31/711; A61K 38/17; A61P 15/18

(52) U.S. Cl. 424/139.1; 514/44 R; 514/12

(57) ABSTRACT

The present invention relates to novel egg membrane protein receptors and to methods of inhibiting the interaction of sperm proteins with egg proteins. The invention further relates to methods of preventing and inhibiting sperm-egg binding, sperm-egg fusion, and fertilization. The invention further relates to the egg membrane proteins MET and ZEP.

### CROSS REFERENCE TO RELATED APPLICATIONS

This application is entitled to priority pursuant to 35 U.S.C. §119(e) to U.S. Provisional Patent Application Ser. Nos. 60/655,562, filed Feb. 23, 2005, and 60/689,181, filed Jun. 10, 2005, the entire disclosures of which are herein incorporated by reference.

### REFERENCE TO GOVERNMENT GRANT

This invention was supported in part by National Institutes of Health grants U54HD29099 and D43TW/HD00654. The U.S. Government may therefore have certain rights in this invention.

### FIELD OF THE INVENTION

The invention relates to methods of preparing and using novel receptors on eggs, MET and ZEP, which are specific for the sperm proteins SLLP11 and SLLP2, and for homologous proteins. The invention also relates to novel egg proteins. The invention also relates to methods of contraception by inhibiting interaction of sperm proteins with egg proteins.

### BACKGROUND OF THE INVENTION

SLLP1 is a unique non-bacteriolytic, c-lysozyme-like protein recently isolated from human spermatozoa (See PCT application nos. PCT/US01/1716, filed Jan. 19, 2001 and PCT/US04/01240, filed Jan. 16, 2004, the disclosures of which are incorporated herein in their entirety). It is encoded by the gene SPACA3 at locus 17q11.2 and the protein is localized in the acrosome of human spermatozoa. Antisera to SLLP11 blocks binding in the hamster egg penetration assay, suggesting that SLLP1 is involved in sperm/egg adhesion.

Lysozyme is known as a powerful antibacterial protein found in many organisms, including invertebrates, plants, fish, birds, and mammals. Within the animal phyla, three main lysozyme types are recognized: chicken-type (c), goose-type, and invertebrate type. Lysozyme belongs to a class of enzymes (muramidase, EC 3.2.1.17) that lyses the cell walls of certain Gram positive bacteria by catalyzing the hydrolysis of the beta-1,4-glycosidic linkage between N-acetylmuramic acid and N-acetylglucosamine in the bacteria cell wall peptidoglycan. It has been suggested that lysozymes, not only protect hosts against infection from invading microorganisms, but also play a role in releasing nutrients from some bacteria in the digestive tract. The potent bacteriolytic lysozyme, present in many tissues and body fluids in human and mouse, is the c-type lysozyme, encoded by a gene located at 12q14.3 in humans and on chromosome 10 in mouse. Conventional c lysozymes possess a substrate binding domain, which accommodates oligosaccharides of N-acetylglucosamine and alternating polymers of N-acetylmuramic acid and N-acetylglucosamine.

During fertilization in mammals, only capacitated spermatozoa are able to penetrate the oocyte. Spermatozoa that reach the zona pellucida receive a signal to undergo the acrosome reaction, an exocytotic event that releases enzymes which appear to facilitate the binding of the sperm to the zona pellucida. Although acrosomal enzymes have been also considered to facilitate hydrolysis of a fertilization channel through the zona pellucida, recent analyses suggest that zona penetration could also be based on the cutting thrust of the oscillating spermatozoa. Upon emergence from the zona pellucida, acrosome-reacted spermatozoa cross the perivitelline matrix (space) and bind to and fuse with the oolemma. Only acrosome-reacted sperm are found in the perivitelline matrix, and only acrosome-reacted sperm are fusogenic, with the plasmalemma domain overlying the equatorial segment thought to mediate oolemma binding and fusion events. Thus, fertilization is completed through direct interactions between sperm and oocyte surface proteins.

A widely discussed model in mammalian fertilization has been binding of a sperm ADAM (A Disintegrin And Metalloprotease) to an egg integrin as a required step for sperm-egg membrane fusion. However, targeted deletion of ADAM2 (fertilin β) and ADAM3 (cyritestin) in mice produces sperm that do not fertilize in vivo but can still normally fuse with eggs in vitro, suggesting that other receptor-ligand interactions may mediate sperm-egg binding and fusion. On the egg, the observation that PI-PLC treatment of mouse eggs but not sperm prevents sperm-oolemma binding and fusion confirms the importance of oolemmal GPI anchored proteins in events at the oolemma. Similarly, conditional knockout of the Pig-a gene, which encodes an enzyme involved in GPI anchor biosynthesis, results in infertile female mice. Furthermore, antibodies to the egg surface tetraspanin, CD9, block fertilization, and CD9 knockout mice are infertile. Currently unknown are the sperm ligands that interact with CD9 or with the class of GPI anchored egg receptors. A recent study indicates that pregnancy-specific glycoprotein, PSG17, a member of the immunoglobulin superfamily, is a ligand for CD9. However, further work should reveal whether this specific ligand is detected on the surface of spermatozoa.

There is a long felt need in the art for methods to block sperm and egg binding and fertilization. The present invention satisfies these needs.

### SUMMARY

The present invention is directed to receptors located on the egg which are specific for sperm SLLP proteins, or for similar proteins. The present invention also encompasses targeting SLLP1, SLLP2, and similar proteins, as well as their receptors, for methods of contraception. The present invention also provides targeting the egg membrane proteins disclosed herein to inhibit or prevent sperm-egg binding, sperm-egg fusion, and fertilization. In one embodiment, the egg proteins are ZEP and MET.

The present invention provides compositions and methods for inhibiting or preventing sperm-egg binding, sperm-egg fusion, and fertilization. In one aspect, the invention provides compositions and methods to block SLLP1 and SLLP2 from binding with an egg or interacting with an egg. In one aspect, compositions and methods are provided for preventing or inhibiting SLLP1 and SLLP2 from binding or interacting with an egg protein. In one embodiment, the egg protein is ZEP. In another embodiment, the egg protein is MET.

In one embodiment, a SLLP protein is prevented or inhibited from interacting with an egg by using an antibody. In one aspect, the antibody is directed against the SLLP protein. In another aspect, the antibody is directed against an egg protein.

In another embodiment, an SLLP protein is prevented or inhibited from interacting with an egg by contacting the sperm or egg by contacting the sperm or egg with a pharmaceutical composition comprising an effective amount of a protein and a pharmaceutically-acceptable carrier. In one aspect, the protein is an SLLP protein. In one aspect, the SLLP protein is SLLP1 or SLLP2, or both, or a homolog, fragment, derivative, or modification thereof. In one aspect, the protein is a recombinant protein. In another aspect, the protein which prevents or inhibits an SLLP protein from interacting with an egg is an isolated egg protein. In one aspect, the egg protein is MET or ZEP, or both, or a homolog, fragment, derivative, or modification thereof. In another aspect, the isolated egg protein is a recombinant protein.

In one embodiment, MET or ZEP is prevented or inhibited from interacting with a sperm by contacting the sperm or an egg with a pharmaceutical composition comprising an effective amount of a compound to prevent or inhibit such interaction, and a pharmaceutically-acceptable carrier. In one aspect, the compound is MET or ZEP, or a homolog, fragment, derivative, or modification thereof. In another aspect, the compound is an antibody directed against MET or ZEP, or an antibody directed against a sperm protein which interacts with MET or ZEP. In one aspect, the compound is an inhibitor of MET or ZEP synthesis, expression, or function.

In one embodiment, the sperm and egg are human.

The invention provides isolated nucleic acids comprising nucleic acid sequences encoding SLLP1, SLLP, MET and ZEP proteins, and variants, homologs, fragments, derivatives, and modifications thereof.

The invention provides amino acid sequences for SLLP1, SLLP2, MET, and ZEP proteins, and variants, homologs, fragments, derivatives, and modifications thereof. In one aspect, the variants, homologs, fragments, derivatives, and modifications thereof are biologically active.

The invention provides pharmaceutical compositions comprising at least one isolated nucleic acid of the invention, and a pharmaceutically-acceptable carrier. The invention further provides pharmaceutical compositions comprising at least one protein, and variants, homologs, fragments, derivatives, and modifications thereof, and a pharmaceutically-acceptable carrier.

In one aspect, the invention provides compositions and methods for preventing fertilization in vitro. In another aspect, the invention provides compositions and methods for preventing and inhibiting fertilization in vivo.

In one embodiment, the invention provides compositions and methods useful as contraceptives. In one aspect, the composition and methods are useful as contraceptives in humans.

In another embodiment, the present invention provides compositions and methods useful as contraceptive vaccines. In one aspect, the invention provides a contraceptive vaccine, said vaccine comprising a pharmaceutical composition of the invention.

The present invention provides nucleic acid and amino acid sequences for mouse and human SLLP1, SLLP2, MET, and ZEP as follows: SEQ ID NO:1—mouse MET normal nucleic acid sequence; SEQ ID NO:2—mouse MET normal amino acid sequence; SEQ ID NO:3—mouse MET variant nucleic acid sequence; SEQ ID NO:4—mouse MET variant amino acid sequence; SEQ ID NO:5—mouse ZEP normal nucleic acid sequence; SEQ ID NO:6—mouse ZEP normal amino acid sequence; SEQ ID NO:7—mouse ZEP variant 1 nucleic acid sequence; SEQ ID NO:8—mouse ZEP variant 1 amino acid sequence; SEQ ID NO:9—mouse ZEP variant 2 nucleic acid sequence; SEQ ID NO:10-mouse ZEP variant 2 amino acid sequence; SEQ ID NO:11—mouse SLLP1 nucleic acid sequence; SEQ ID NO:12—mouse SLLP1 amino acid sequence; SEQ ID NO:13—human SLLP1 nucleic acid sequence; SEQ ID NO:14—human SLLP1 amino acid sequence; SEQ ID NO:15—mouse SLLP2 nucleic acid sequence; SEQ ID NO:16—mouse SLLP2 mature protein amino acid sequence; SEQ ID NO:17—human SLLP2 nucleic acid sequence; and SEQ ID NO: 18—human SLLP2 amino acid sequence.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1: Deduced amino acid sequence of the full-length mSLLP1. A putative transmembrane domain (71 to 92 aa) and a putative protease cleavage site are designated with a rectangular box and with a downward arrow, respectively. The presence of an alpha-lactalbumin/lysozyme C signature sequence is marked with underline. The altered residues in SLLP1 considered essential to the catalytic pocket of c lysozymes are shown in bold and underlined while the three putative myristoylation sites are circled. The multiple putative phosphorylation sites are shown in boxes. The GenBank accession number of this sequence is AK006357. The numbers on the left and right side of the sequence indicate the residue positions beginning with translation start site and after the protease cleavage site, respectively.

FIG. 2: Multiple amino acid sequence alignment of mature SLLP1 (mouse and human) with human, mouse and chicken c-type lysozymes. Identical residues were shaded while the 20 invariant residues of c lysozymes were indicated in bold in the consensus sequence. The four non-conserved invariant residues of mSLLP1 were underlined in the consensus sequence. The codons of the boxed residues were interrupted by the introns. The residues marked with diamonds form a part of the considered ligand binding domain in c-type lysozymes with five out of these six residues being conserved in both mouse and human SLLP1. The arrowheads indicate two critical residues (E35 and D52) responsible for the catalytic activity of chicken lysozyme that are mutated in mouse and human SLLP1. Numbers above the top sequence refer to amino acid positions relative to the first residue. Percent identity and similarity of mSLLP1 with hSLLP1 and c-type lysozymes are shown at the end of each sequence. Lz: lysozyme; mSLLP1: mouse sperm lysozyme-like protein (AAT07446); hSLLP1: human sperm lysozyme-like protein 1 (AAK01478); hLZ: human lysozyme (P00695); mLz: mouse lysozyme (P08905); cLz: chicken egg white lysozyme (LZCH).

FIG. 3: Profile of purified recmSLLP1 and specificity of its antisera. (A) Expression and purification of his-tagged mature recmSLLP1 (˜15 kDa) in BL21-DE3 cells stained with silver (left panel) or anti-his antibody (right panel). Left panel: lane 1: molecular weigh markers, lane 2: extracts from uninduced cells, lane 3: extracts following induction with IPTG for 3 h, lane 4: 1 μg of affinity purified recmSLLP1. Right panel: lane 1:1 μg affinity purified recmSLLP1, lane 2: 3 h induced extract, lane 3: uninduced extract, lane 4: molecular weigh markers. (B) Specificity of the antisera generated in female guinea pigs against purified recombinant mature mSLLP1 (left panel) and cauda epididymal mouse sperm extract (right panel). Left panel: lane 1: serum from adjuvant injected controls (1:2,000), lane 2: preimmune (1:2,000), lane 3: immune 1:15,000, lane 4: immune 1:30,000. Right panel: lane 1: serum from adjuvant injected controls (1:2,000), lane 2: preimmune (1:2,000), lane 3: immune 1:5,000, lane 4: immune 1:10,000. Neither preimmune nor serum from animals injected with the adjuvant alone reacted with the recombinant protein (0.1 μg/lane, left panel) or with mouse sperm proteins (10 μg/lane, right panel), while the anti-recmSLLP1 serum was reactive with recmSLLP1 and with a ˜14 kDa mouse sperm protein. Arrows indicate reactive recombinant protein at ˜15 kDa and the dimer formed at ˜30 kDa.

FIG. 4: Indirect immunofluorescent localization of mSLLP1 on acrosome intact mouse spermatozoa. Phase-contrast (A, C, G), anti-mSLLP1 (B, D, H), acrosome staining with Peanut agglutinin (PNA) lectin (E) and double staining of acrosome intact sperm with PNA and anti-recmSLLP1 serum (F). (A) and (B) are insets of (C) and (D) respectively. SLLP1 was localized mainly to the anterior acrosome (arrowheads), with some sperm showing staining in the equatorial segment (arrow). Acrosome-intact sperm defined by the presence of PNA staining on the acrosome (E) showed staining with recmSLLP1 antibody (F) indicating that mSLLP1 is located in the acrosome of non-capacitated mouse sperm. No immunostaining was observed with preimmune sera (H).

FIG. 5: Localization of mSLLP1 in ionophore-induced acrosome reacted sperm. Phase contrast image (1) may be compared to immunofluorescence with anti-recmSLLP1 (A) and to acrosome-reacted sperm identified by the absence of PNA fluorescence (C). SLLP1 was identified in the equatorial segment (arrowhead) in the majority of acrosome reacted mouse sperm. The inset in A is a magnified view of the spermatozoon with arrowhead.

FIG. 6: Confocal immunofluorescent localization of mouse SLLP1 on spermatozoa tightly bound to the mouse oolemma. Zona-free eggs inseminated with capacitated spermatozoa were fixed and stained with anti-recmSLLP1 serum and incubated with Sytox for detection of nuclear staining and examined on a Zeiss 410 Axiovert 100 microsystem. (A) Phase-contrast; (B) nuclear DNA staining; (C) anti-recmSLLP1 immune and (D) overlay of B and C. Immune sera revealed mSLLP1 staining in sperm bound to the egg (arrow). When sperm were imaged in their respective focal planes (32 sperm from 3 eggs), all sperm tightly bound to the egg displayed mSLLP1 staining. The insert in panel D is an enlarged view of one sperm bound to the oolemma (indicated with arrow in D).

FIG. 7: Effect of anti-SLLP1 antibody (A) or recmSLLP1 (B) on sperm-egg binding and fusion. Zona pellucidae from mature mouse eggs were removed by brief incubation in chymotrypsin followed by mechanical shearing. (A) Capacitated mouse sperm, pre-incubated with different concentrations of anti-recmSLLP1 sera (solid bars) were co-incubated with zona-free mouse eggs. (B) Zona-free eggs were pre-incubated with the indicated concentrations (μg/ml) of recmSLLP1 and then inseminated with capacitated mouse sperm. In all cases, the sera or the recombinant proteins were present during gamete interaction. Eggs were processed and analyzed for sperm binding and fusion. Data represent the mean±SE from five different experiments. (*) P≦0.05; (**) P≦0.01. The numbers above the bars represent the number of eggs per group. Anti-recmSLLP1 sera as well as the recmSLLP1 protein significantly inhibited sperm-egg binding and fusion to the oolemma in a concentration-dependent manner. Controls: (A) preimmune sera, open bars; (B) no protein buffer (cont.).

FIG. 8: Indirect immunofluorescence of mSLLP1 complementary binding sites on the egg surface. (A) Mature unfertilized or (B) fertilized mouse eggs, with (i) or without (ii) zona pellucida were incubated with 100 μg/ml recmSLLP1 for 45 min, washed, exposed to anti-recmSLLP1 sera followed by goat anti-guinea pig/FITC antisera. To visualize nuclear DNA eggs were treated with 1 μM Hoechst 33342 for 10 min and then washed. Mouse SLLP1 binding sites were localized in the perivitelline space and on the microvillar region of unfertilized oocytes. Similar binding pattern was also observed with 200, 50, 10, 1.0 and 0.1 μg/ml of final mSLLP1 concentration to the zona intact and zona free unfertilized mouse eggs (data not shown). In fertilized eggs staining was observed along the entire plasma membrane of both zona free and zona intact oocytes. Arrows indicate specific fluorescent staining. Note the metaphase staining (A-i, ii, lower panels, in purple) and pronuclear staining (B-i, ii, lower panels). (C) Control groups: unfertilized oocytes with or without zona were incubated in the absence of recmSLLP1 (i), or in presence of recmSLLP1 but with pre-immune sera (ii), or in the presence of 200 μg/ml recePAD (iii). (ZP): zona pellucida; (PVS): perivitelline space.

FIG. 9: SLLP1 localization by scanning confocal analysis of metaphase II oocytes (A) and pronuclear stage embryos (B) incubated with 100 μg/ml recmSLLP1, followed by anti-recmSLLP1 immune sera and donkey anti-guinea pig/Texas red secondary antibody. The green fluorescence corresponds to the nuclear Sytox DNA stain. Intense red immunofluorescences localized SLLP1 throughout the perivitelline space of unfertilized oocytes (A). An intense but discontinuous distribution of immunofluorescence was detected over the entire surface of fertilized eggs (B, white arrowheads). Note that SLLP1 was also localized in the zona pellucidae (asterisks) of both metaphase II and fertilized eggs although the fluorescent signal is much reduced from that seen at the perivitelline space or oolemma (A, B). No immunostaining was observed with recmSLLP1+preimmune sera (C) or with recmSLLP1+second antibody alone (D).

FIG. 10: Comparison of mouse and human SLLP1 with conventional c lysozymes binding to mouse eggs. (A) Effect of chicken and human c lysozymes on sperm-egg binding and fusion. Zona-free eggs were pre-incubated with chicken or human lysozyme (50 or 100 μg/ml) or with mouse (50 μg/ml) or human (25 μg/ml) SLLP1 and then inseminated with capacitated mouse sperm. Note that both lysozymes as well as SLLP1 were present during fertilization. Eggs were processed and analyzed for sperm binding and fusion. Data represent the mean±SE from three different experiments. (**) P≦0.01. The numbers above the bars represent the number of oocytes per group. Neither chicken nor human lysozyme was able to block sperm-egg binding or fusion. Controls: no protein added; 50 μg/ml recmSLLP1; 25 μg/ml rechSLLP1 (positive controls). (13) Indirect immunofluorescence of mouse eggs incubated with c lysozymes to determine whether conventional lysozymes can bind to the egg surface in a manner comparable to SLLP1. Unfertilized mouse eggs, with or without zona pellucida were incubated with 100 μg/ml chicken (a) or human (b) lysozyme, washed, exposed to anti-chicken lysozyme (1:400) and anti-human lysozyme (1:25) antibodies respectively, followed by FITC-conjugated secondary antibodies (1:200). In comparison, no egg binding of either conventional lysozyme was detected on metaphase II mouse eggs. (c) 100 μg/ml recmSLLP1+anti-recmSLLP1 immune sera (1:50) was used as a positive control in which mSLLP1 showed characteristic binding to microvillar region and perivitelline space.

FIG. 11, comprising FIGS. 11A and B, graphically depicts the effects of recombinant mouse SLLP1 protein on the binding of sperm to eggs (A) and on the fusion (B) of sperm with eggs.

FIG. 12, schematically depicts human and mouse SLLP1 and SLLP2 peptides.

FIG. 13, schematically depicts human SLLP2 cDNA and deduced amino acid sequences.

FIG. 14, comprising FIGS. 14A and 14B, depicts images of analyses showing that human SLLP2 is expressed specifically in testis (sperm).

FIG. 15, comprising FIGS. 15A (pre-immune) and B (immune), represents images of an immuno-electron microscopic analysis localizing the expression of human SLLP2 to the acrosomal region of ejaculated human sperm.

FIG. 16 is a schematic representation comparing and aligning human SLLP2 protein sequence with that of homologues, including lysozymes and human SLLP1.

FIG. 17, comprising A-F, represent images of photographs showing that human SLLP2 binds to mouse eggs. Upper panel (A-C)—+hSLLP2; Lower panel (D-F)—no hSLLP2.

FIG. 18, comprising eight photographs (left panel—A-D, no SLLP2; right panel—A-D, +SLLP2), demonstrates that mouse SLLP2 binds to zona intact mouse eggs.

FIG. 19, comprising eight photographs (left panel—A-D, no SLLP2; right panel—A-D, +SLLP2), demonstrates that mouse SLLP2 binds to zona free mouse eggs.

FIG. 20 is a graphic representation of the results of an experiment demonstrating that recombinant mouse SLLP2 inhibits mouse sperm from binding with mouse eggs.

FIG. 21 is a graphic representation of the results of an experiment demonstrating that recombinant mouse SLLP2 inhibits mouse sperm from fusing with mouse eggs.

FIG. 22 is a graphic representation of the results of an experiment demonstrating that recombinant mouse SLLP2 inhibits mouse sperm from fertilizing mouse eggs. The six groups, from left to right, are 200, 100, 50, and 25 μg/ml recombinant mouse SLLP2, BSA at 200 μg/ml, and PBS. The ordinate represents percentage of fertilization.

FIG. 23 is a schematic representation of the conservation of the sequence of human SLLP2 in mammals.

FIG. 24 is a schematic representation of the dog ortholog of human SLLP2 and the conservation of the sequence.

FIG. 25 is a schematic representation and alignment of the human MET protein and its deletion variant.

FIG. 26, comprising left and right panels, represents images of an electrophoretic analysis of recombinant MET variants in E. coli. A-MET; B-MET-V.

FIG. 27 represents an image of an electrophoretic analysis of the purification of induced MET on an His-binding affinity column. U—uninduced; I—induced

FIG. 28 is a schematic representation of the MET protein sequence, depicting the alanine rich domain (underlined).

FIG. 29 represents an image of a western blot analysis of recombinant MET reacted with a 50,000 dilution of immune sera. PI—preimmune; I—immune

FIG. 30 represents an image of an immune sera screening analysis using 50 ng of bacterial recombinant MET.

FIG. 31, comprising images of six photomicrographs, represents images of the immuno-localization of MET in sections of ovary. Upper panel (IM—immune); Lower panel (PI—preimmune).

FIG. 32, comprising images of six photomicrographs, represents images of the immunolocalization of MET on zona intact live eggs. The upper panel represents IM—immune, while the lower panel represent PI.

FIG. 33, comprising images of 28 photomicrographs, represents images of the immunolocalization of MET during various embryonic developmental stages. The upper panel (upper two rows) represents I—immune treated groups; while the lower panel (lower two rows) represents pre-immune sera treated groups.

FIG. 34, comprising images A and B, demonstrates images of a farwestern analysis (A and B) showing the protein interaction of MET and SLLP1. MET was electrophoresed, the gel was transferred to nitrocellulose, and then overlaid (OL) with recombinant SLLP1. U—uninduced; I—induced; P—purified; +OL—overlaid; −OL—no overlay.

FIG. 35 is a schematic representation of the amino acid sequence of the zinc endopeptidase, ZEP protein, and its alignment with its variants, ZEP-V1 and ZEP-V2. N—normal; V1—variant 1; V2—variant 2.

FIG. 36, comprising A-C, represents images of an electrophoretic analysis comparing ZEP protein (ZP) and its two variants (ZP-V1 and ZP-V2). ZP—normal ZEP; ZP-V1—variant 1 of ZEP; ZP-V2—variant 2 of ZEP.

FIG. 37 represents an image of an electrophoretic analysis of induced zin-endopeptidase (ZEP) purified on a His-binding affinity column. Lane 1—standard markers; Lane 2—uninduced cells (U); Lane 3—induced cells (1); Lane 4—purified ZEP from induced cells (P).

FIG. 38 is a schematic representation of the amino acid sequence of ZEP, indicating the predicted transmembrane domain, cleavage site, and a zinc binding signature.

FIG. 39 represents an image of a western blot of recombinant ZEP (50 ng) reacted with a 50,000 dilution of immune sera (lane 3).

FIG. 40 represents an image of an electrophoretic analysis in which immune sera prepared against ZEP was used in a western blot to detect ZEP in zona intact mouse eggs (ZIE) and zona free mouse eggs (ZFE). The left panel indicates preimmune sera (PI) and the right panel indicates immune sera (1).

FIG. 41, comprising six panels, represents images of photographs taken in an immunolocalization study of ZEP in sections of ovary. Upper row—immune sera (IM); Lower row—preimmune sera (PI).

FIG. 42, comprising FIGS. 42a and 42b, represents images of immunolocalization studies of ZEP on zona intact eggs (42a) and on zona free eggs (42b). Each figure comprises four images. The upper two images of each were subjected to immune sera (IM), while the lower images of each were subjected to preimmune sera (PI).

FIG. 43, comprising 28 images, represents images of an immunolocalization analysis of ZEP during early embryonic stages. The stages include, from left to right, germinal vesicle (GV), M2, PN, 2 cell, 4 cell, morula, and blastocyst. Phase contrast (upper images) and fluorescent (lower images) are matched for groups subjected to immune or preimmune sera.

FIG. 44, comprising four images, represents confocal images in which ZEP and the sperm protein SLLP1 are co-localized in mouse eggs exposed to purified SLLP1. The left image represents a micrograph of the treated egg, the second image indicates localization of ZEP, the third image indicates localization of SLLP1 bound to the egg, and the fourth image is a composite image where the ZEP and SLLP1 images are merged.

FIG. 45, comprising A and B, illustrates a far-western analysis to demonstrate that the sperm protein SLLP1 binds with the egg protein ZEP. Panel A represents an image of an electrophoretic analysis of a cell comprising ZEP in an uninduced (U) state, induced (1), and the purified ZEP protein from an induced cell (P). The left lane of panel A indicates the standard/molecular weight markers. Panel B represents an image where purified egg ZEP had been electrophoresed, transferred to nitrocellulose, and then overlaid (OL) or not (−OL) with recombinant sperm SLLP1, and then probed with an anti-SLLP1 monoclonal antibody.

Other aspects and advantages of the present invention are described herein and in the following detailed description of the preferred embodiments thereof.

### DETAILED DESCRIPTION OF THE INVENTION

### Abbreviations and Acronyms

BSA means bovine serum albumin
GV means germinal vesicle
h means human
I means induced or immune
IM means immune
m means mouse
MET means mouse egg-specific TolA (referred to in the provisional application as a Colcin-like uptake protein or Colicin uptake protein)
OL means overlay
P means purified
PI means pre-immune
PBS means phosphate-buffered saline
rec means recombinant, as does “r”
SLLP means sperm lysozyme-like protein
U means uninduced
ZEP means zinc endopeptidase (referred to in the provisional application as zinc peptidase, or ZP)
ZFE means zona free egg
ZIE means zona intact egg

### SEQ ID NOs:

SEQ ID NO:1—mouse MET normal nucleic acid sequence
SEQ ID NO:2—mouse MET normal amino acid sequence
SEQ ID NO:3—mouse MET variant nucleic acid sequence
SEQ ID NO:4—mouse MET variant amino acid sequence
SEQ ID NO:5—mouse ZEP normal nucleic acid sequence
SEQ ID NO:6—mouse ZEP normal amino acid sequence
SEQ ID NO:7—mouse ZEP variant 1 nucleic acid sequence
SEQ ID NO:8—mouse ZEP variant 1 amino acid sequence
SEQ ID NO:9—mouse ZEP variant 2 nucleic acid sequence
SEQ ID NO:10—mouse ZEP variant 2 amino acid sequence
SEQ ID NO:11—mouse SLLP1 nucleic acid sequence
SEQ ID NO:12—mouse SLLP1 amino acid sequence
SEQ ID NO:13—human SLLP1 nucleic acid sequence
SEQ ID NO:14—human SLLP1 amino acid sequence
SEQ ID NO:15—mouse SLLP2 nucleic acid sequence
SEQ ID NO:16—mouse SLLP2 mature protein amino acid sequence
SEQ ID NO:17—human SLLP2 nucleic acid sequence
SEQ ID NO:18—human SLLP2 amino acid sequence

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described herein. In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

A disease, disorder, or condition is “alleviated” if the severity of a symptom of the disease or disorder, the frequency with which such a symptom is experienced by a patient, or both, are reduced.

As used herein, “amino acids” are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

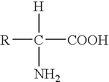

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

As used herein, an “analog” of a chemical compound is a compound that, by way of example, resembles another in structure but is not necessarily an isomer (e.g., 5-fluorouracil is an analog of thymine).

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “antisense oligonucleotide” means a nucleic acid polymer, at least a portion of which is complementary to a nucleic acid which is present in a normal cell or in an affected cell. The antisense oligonucleotides of the invention include, but are not limited to, phosphorothioate oligonucleotides and other modifications of oligonucleotides. Methods for synthesizing oligonucleotides, phosphorothioate oligonucleotides, and otherwise modified oligonucleotides are well known in the art (U.S. Pat. No. 5,034,506; Nielsen et al., 1991, Science 254: 1497). “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

The term “basic” or “positively charged” amino acid as used herein, refers to amino acids in which the R groups have a net positive charge at pH 7.0, and include, but are not limited to, the standard amino acids lysine, arginine, and histidine.

The term “biocompatible”, as used herein, refers to a material that does not elicit a substantial detrimental response in the host.

As used herein, the term “biologically active fragments” or “bioactive fragment” of the polypeptides encompasses natural or synthetic portions of the full-length protein that are capable of specific binding to their natural ligand or of performing the function of the protein.

“C19” and “C23” are names which are also used for “SLLP1” and SLLP2”, respectively.

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.”

Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to a protein, polypeptide, an isolated nucleic acid, or other agent used in the method of the invention.

As used herein, the term “conservative amino acid substitution” is defined herein as an amino acid exchange within one of the following five groups:

| | |
|---|---|
| I. | Small aliphatic, nonpolar or slightly polar |
| | residues: |
| | Ala, Ser, Thr, Pro, Gly; |
| II. | Polar, negatively charged residues and their |
| | amides: |
| | Asp, Asn, Glu, Gln; |
| III. | Polar, positively charged residues: |
| | His, Arg, Lys; |
| IV. | Large, aliphatic, nonpolar residues: |
| | Met Leu, Ile, Val, Cys |
| V. | Large, aromatic residues: |
| | Phe, Tyr, Trp |

“Contraceptive”, as used herein, refers to an agent, compound, or method that diminishes the likelihood of or prevents conception.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cell are present in the tissue in an animal not afflicted with a disease or disorder.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the phrases “egg protein” or “egg-specific protein” refer to proteins which are expressed exclusively or predominately in eggs or ovaries. The proteins need not be expressed at all stages of egg or ovarian development.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “enhancer” is a DNA regulatory element that can increase the efficiency of transcription, regardless of the distance or orientation of the enhancer relative to the start site of transcription.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, a “functional” biological molecule is a biological molecule in a form in which it exhibits a property or activity by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

By the term “immunizing a subject against an antigen” is meant, administering to the subject a composition, a protein complex, a DNA encoding a protein complex, an antibody or a DNA encoding an antibody, which elicits an immune response in the subject, which immune response provides protection to the subject against the condition caused by the antigen or related to the presence of the antigen.

The term “inhibit,” as used herein, refers to the ability of a compound of the invention to reduce or impede a described function. Preferably, inhibition is by at least 10%, more preferably by at least 25%, even more preferably by at least 50%, and most preferably, the function is inhibited by at least 75%.

The phrase “inhibit conception”, as used herein, refers to both direct and indirect inhibition of conception or impregnation, regardless of the mechanism. The phrase also includes reducing the rate of conception, and does not necessarily mean that conception is inhibited by 100%.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

By “interaction” between a sperm protein and an egg protein is meant the interaction such as binding which is necessary for an event or process to occur, such as sperm-egg binding, fusion, or fertilization. In one aspect, the “interaction” may be similar to receptor-ligand type of binding or interaction.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow and Lane, 1988, Antibodies, A Laboratory Manual, Cold Spring Harbor Publications, New York, for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Operably linked” refers to a juxtaposition wherein the components are configured so as to perform their usual function. Thus, control sequences or promoters operably linked to a coding sequence are capable of effecting the expression of the coding sequence. By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, a “peptide” encompasses a sequence of 2 or more amino acid residues wherein the amino acids are naturally occurring or synthetic (non-naturally occurring) amino acids covalently linked by peptide bonds. No limitation is placed on the number of amino acid residues which can comprise a protein's or peptide's sequence. As used herein, the terms “peptide,” polypeptide,” and “protein” are used interchangeably. Peptide mimetics include peptides having one or more of the following modifications:

1. peptides wherein one or more of the peptidyl —C(O)NR— linkages (bonds) have been replaced by a non-peptidyl linkage such as a—CH₂-carbamate linkage (—CH₂OC(O)NR—), a phosphonate linkage, a —CH₂-sulfonamide (—CH₂—S(O)₂NR—) linkage, a urea (—NHC(O)NH—) linkage, a—CH₂-secondary amine linkage, or with an alkylated peptidyl linkage (—C(O)NR—) wherein R is C₁-C₄ alkyl;

2. peptides wherein the N-terminus is derivatized to a —NRR₁ group, to a—NRC(O)R group, to a —NRC(O)OR group, to a—NRS(O)₂R group, to a—NHC(O)NHR group where R and R₁ are hydrogen or C₁-C₄ alkyl with the proviso that R and R₁ are not both hydrogen;

3. peptides wherein the C terminus is derivatized to —C(O)R₂ where R₂ is selected from the group consisting of C₁-C₄ alkoxy, and —NR₃R₄ where R₃ and R₄ are independently selected from the group consisting of hydrogen and C₁-C₄ alkyl.

Synthetic or non-naturally occurring amino acids refer to amino acids which do not naturally occur in vivo but which, nevertheless, can be incorporated into the peptide structures described herein. The resulting “synthetic peptide” contains amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydrox, phenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents.

The term also encompasses any of the agents approved by a regulatory agency of the US Federal government or listed in the US Pharmacopeia for use in animals, including humans.

A “polylinker” is a nucleic acid sequence that comprises a series of three or more different restriction endonuclease recognitions sequences closely spaced to one another (i.e. less than 10 nucleotides between each site).

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “non-native promoter” as used herein refers to any promoter that has been operably linked to a coding sequence wherein the coding sequence and the promoter are not naturally associated (i.e. a recombinant promoter/coding sequence construct).

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Plurality” means at least two.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA.

“Recombinant polynucleotide” refers to a polynucleotide having sequences that are not naturally joined together. An amplified or assembled recombinant polynucleotide may be included in a suitable vector, and the vector can be used to transform a suitable host cell. A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

A “reversibly implantable” device is one which may be inserted (e.g. surgically or by insertion into a natural orifice of the animal) into the body of an animal and thereafter removed without great harm to the health of the animal.

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody). By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

“SLLP1” and SLLP2” are also referred to as “C19” and “C23”, respectively.

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds,” as used herein, is meant an antibody or compound which recognizes and binds a molecule of interest (e.g., an antibody directed against a polypeptide of the invention), but does not substantially recognize or bind other molecules in a sample.

The term “standard,” as used herein, refers to something used for comparison. For example, a standard can be a known standard agent or compound which is administered or added to a control sample and used for comparing results when measuring said compound in a test sample. Standard can also refer to an “internal standard,” such as an agent or compound which is added at known amounts to a sample and is useful in determining such things as purification or recovery rates when a sample is processed or subjected to purification or extraction procedures before a marker of interest is measured.

A “subject” of analysis, diagnosis, or treatment is an animal. Such animals include mammals.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C. with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “transgenic mammal” means a mammal, the germ cells of which comprise an exogenous nucleic acid.

As used herein, a “transgenic cell” is any cell that comprises a nucleic acid sequence that has been introduced into the cell in a manner that allows expression of a gene encoded by the introduced nucleic acid sequence.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease. As used herein, the term “treating” includes alleviating the symptoms associated with a specific disease, disorder or condition and/or preventing or eliminating said symptoms.

By the term “vaccine,” as used herein, is meant a composition which when inoculated into a subject has the effect of stimulating an immune response in the subject, which serves to fully or partially protect the subject against a condition, disease or its symptoms. In one aspect, the condition is conception. The term vaccine encompasses prophylactic as well as therapeutic vaccines. A combination vaccine is one which combines two or more vaccines, or two or more compounds or agents.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, plasmids, cosmids, lambda phage vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

In one embodiment, the invention provides a pharmaceutical composition comprising a pharmaceutically-acceptable carrier and at least one egg protein, or a homolog, fragment or derivative thereof, wherein said protein is capable of inducing an immune response useful for inhibiting conception in a subject. In one aspect, the invention provides a pharmaceutical composition, wherein said egg protein comprises an amino acid sequence selected from the group consisting of SEQ ID NOs:2, 4, 6, 8, 10. In another aspect, the invention provides a pharmaceutical composition further comprising at least one additional egg protein, wherein said known egg protein is capable of inducing an immune response useful for contraception.

In one embodiment, the invention provides a pharmaceutical composition, wherein said pharmaceutical composition comprises at least two different proteins, or homologs, fragments, or derivatives thereof, wherein said at least two different proteins comprise an amino acid sequence selected from the group consisting of SEQ ID NOs: 2, 4, 6, 8, 10.

In one embodiment, the invention provides a contraceptive vaccine, said vaccine comprising a pharmaceutical composition of the invention, comprising one or more proteins, or variants, homologs, or fragments thereof, comprising an amino acid sequence selected from the group consisting of SEQ ID NOs: 2, 4, 6, 8, and 10, and at least one other egg protein, or a variant, fragment, or homolog thereof.

In one embodiment, the invention provides a method for inhibiting conception in a subject, said method comprising administering to said subject a pharmaceutical composition comprising a pharmaceutically-acceptable carrier and at least one egg protein, or a homolog, fragment or derivative thereof, wherein said protein is capable of inducing an immune response useful for inhibiting conception in a subject. In one aspect, the egg protein comprises an amino acid sequence selected from the group consisting of SEQ ID NOs: 2, 4, 6, 8, 10.

It will be appreciated, of course, that the proteins or peptides of the invention may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Nucleic acids useful in the present invention include, by way of example and not limitation, oligonucleotides and polynucleotides such as antisense DNAs and/or RNAs; ribozymes; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, double-stranded DNA, supercoiled DNA and/or triple-helical DNA; Z-DNA; and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, OLIGONUCLEOTIDE SYNTHESIS: A PRACTICAL APPROACH (IRL Press, Oxford, England)). RNAs may be produce in high yield via in vitro transcription using plasmids such as SP65 (Promega Corporation, Madison, Wis.).

The peptides of the present invention may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the proteins or peptides obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide can be purified to remove contaminants. In this regard, it will be appreciated that the peptide will be purified to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure peptide obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

The present invention is also directed to pharmaceutical compositions comprising the compounds of the present invention. More particularly, such compounds can be formulated as pharmaceutical compositions using standard pharmaceutically acceptable carriers, fillers, solublizing agents and stabilizers known to those skilled in the art.

The invention is also directed to methods of administering the compounds of the invention to a subject. In one embodiment, the invention provides a method of treating a subject by administering compounds identified using the methods of the invention description. Pharmaceutical compositions comprising the present compounds are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

In accordance with one embodiment, a method of treating a subject in need of such treatment is provided. The method comprises administering a pharmaceutical composition comprising at least one compound of the present invention to a subject in need thereof. Compounds identified by the methods of the invention can be administered with known compounds or other medications as well.

The invention also encompasses the use of pharmaceutical compositions of an appropriate compound, and homologs, fragments, analogs, or derivatives thereof to practice the methods of the invention, the composition comprising at least one appropriate compound, and homolog, fragment, analog, or derivative thereof and a pharmaceutically-acceptable carrier.

The pharmaceutical compositions useful for practicing the invention may be administered to deliver a dose of between 1 ng/kg/day and 100 mg/kg/day.

The invention encompasses the preparation and use of pharmaceutical compositions comprising a compound useful for treatment of the diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys. The invention is also contemplated for use in contraception for nuisance animals such as rodents.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically, dosages of the compound of the invention which may be administered to an animal, preferably a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the disease being treated, the type and age of the animal, etc.

The invention also includes a kit comprising the composition of the invention and an instructional material which describes adventitially administering the composition to a cell or a tissue of a mammal. In another embodiment, this kit comprises a (preferably sterile) solvent suitable for dissolving or suspending the composition of the invention prior to administering the compound to the mammal.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviation the diseases or disorders in a cell or a tissue of a mammal. The instructional material of the kit of the invention may, for example, be affixed to a container which contains the peptide of the invention or be shipped together with a container which contains the peptide. Alternatively, the instructional material may be shipped separately from the container with the intention that the instructional material and the compound be used cooperatively by the recipient.

The invention is now described with reference to the following Examples and Embodiments. Without further description, it is believed that one of ordinary skill in the art can, using the preceding description and the following illustrative examples, make and utilize the present invention and practice the claimed methods. The following working examples therefore, are provided for the purpose of illustration only and specifically point out the preferred embodiments of the present invention, and are not to be construed as limiting in any way the remainder of the disclosure. Therefore, the examples should be construed to encompass any and all variations which become evident as a result of the teaching provided herein.

### EXAMPLES

### Example 1

### SLLP1 and its Role in Binding to Eggs

Materials and Methods

Cloning and Expression of Mouse SLLP1 (mSLLP1)

Human SLLP1 and SLLP2 nucleic acids and proteins were previously identified and sequenced by the inventors (see U.S. patent application Ser. No. 10/181,611, filed Jul. 18, 2002, the entirety of which is incorporated herein by reference). These proteins were further characterized, and other members of the family were identified by the present inventors (see U.S. patent application Ser. No. 10/542,038 filed Jul. 13, 2005, the entirety of which is incorporated herein by reference; see also Mandal et al., 2003, Biology of Reproduction, 68:1525-1537, the entirety of which is incorporated herein by reference). Both SLLP proteins are sperm specific in their expression.

Using a Blast search tool (Altschul, 1990), a mouse oithologue of the human SLLP1 was sought in the NCBI GenBank database and a candidate gene identified. Single gene-specific forward and reverse primers with NcoI and XhoI restriction sites respectively were designed to amplify the predicted processed form (128 amino acids, from 94 to 221) of the mouse SLLP1. Primers were obtained from Invitrogen (Carlsbad, Calif.). The cDNA was amplified by PCR from a mouse testis cDNA library (Clontech, Palo Alto, Calif.). The cycling parameters employed were 94° C., 2 min; 94° C., 30 sec; 51° C., 1 min; and 68° C., 1.5 min, for 40 cycles. PCR reaction products were separated on agarose gels, and a band of ˜400 bp was isolated, reamplified, and subcloned in pCR2.1 TOPO vector (Invitrogen). Multiple cDNA clones were sequenced in both directions using vector-derived primers on a Perkin-Elmer Applied Biosystems DNA sequencer (Biomolecular Research Facility, Univ. of Virginia Health System, VA). The cloned cDNA sequence for mouse SLLP1 was submitted to the GenBank (accession number, AY601763). The insert was then restriction digested, gel purified, ligated into the predigested pET28b+ vector and used to transform competent BL21DE3 cells (Novagen, Madison, Wis.). The final construct added two amino acids at the N-terminus and eight residues at the C-terminus including a six histidine tag. A 7.0 ml culture from a single colony was grown to optical density of ˜0.8 at 600ₙₘ at 37° C. in Luria broth (LB) in the presence of 50 μg/ml of kanamycin. Isopropyl-β-D-thiogalactopyranoside (IPTG) (Sigma, St. Louis, Mo.) was then added to a final concentration of 1 mM to induce expression. Following 3 h of induction, the bacteria were collected by centrifugation. The recombinant protein was isolated from the insoluble fraction of E. coli, dissolved in 8 M urea in binding buffer (20 nm tris-HCl, pH 7.9, 5 mM imidazole and 0.5 M NaCl) and purified on a His binding Ni²⁺ chelation affinity resin column by a modification of the manufacture's procedures (Novagen). The eluates were then dialyzed overnight against three changes of PBS. The dialyzed protein was stored at −20° C. until used. Protein concentrations were determined by Coomassie Plus-200 (Pierce, Rockford, Ill.) using bovine serum albumin (BSA) as a standard.

Polyclonal Antibody Production and Western Blot Analysis

Five adult virgin female guinea pigs were used for antibody production against the purified recmSLLP1. Preimmune serum was collected by heart puncture, and subsequently, each animal was injected with 200 μg of the purified recmSLLP1 in complete Freund's adjuvant and boosted twice at intervals of 14 days with the same amount of protein in incomplete Freund's adjuvant. For all immunizations, half of the antigen emulsion was injected intramuscularly in the legs and half subcutaneously in two sites on the back. All animals were exsanguinated by heart puncture 9 days after the final immunization. Blood was collected in serum separation tubes (Becton Dickinson, Franklin Lakes, N.J.). After centrifugation at 1750 g for 10 min, the serum was removed, aliquoted, and frozen until needed.

Specificity of the antisera was tested against recmSLLP1 and mouse sperm extracts following 1D SDS-PAGE western blotting. RecmSLLP1 (0.1 μg/lane) or cauda epididymal mouse spermatozoa (10 μg/lane) were solubilized in Laemmli buffer (2×) and proteins were resolved on a 15% SDS-PAGE gel and separated at 20 mA. Proteins were then blotted to nitrocellulose and stained by Ponceau. All blots were blocked with 5% nonfat dry milk in PBS with 0.05% Tween 20 (PBS-T) for 30 min at room temperature. For immunoblotting of purified recmSLLP1, 1:15,000 or 1:30,000 dilution of the anti-recmSLLP1 guinea pig sera was tested, whereas for mouse sperm proteins, 1:5,000 or 1:10,000 dilutions of the sera was used. The blots were then washed three times for 10 min in PBS-T, and incubated with 1:5000 dilution of peroxidase conjugated goat anti-guinea pig IgG secondary antibody for 1 h and washed two times for 10 min in PBS-T. The blots were then developed either in TMB peroxidase substrate (3,3′,5,5′-tetramethylbenzidine, KPL, Gaithersburg, Md.) or with ECL reagent (Amersham Corp., Buckinghamshire, UK).

Culture Media and Reagents for In Vitro Fertilization Assays

The medium used for in vitro fertilization assays was Fraser's modification of Whittingham's medium (Fraser and Drury, 1975) supplemented with 3% BSA and prepared with culture grade H₂O with analytical-grade reagents. TYH (Toyoda, 1971) medium was used for sperm-oolemma binding assays. Pregnant mare's serum gonadotrophin (PMSG), human chorionic gonadotrophin (hCG), BSA, culture grade H₂O, hyaluronidase, chymotrypsin, Hoechst dye 33342 and other reagents were obtained from Sigma.

Gamete Preparation for In Vitro Assays

Hybrid F1 mice (C57BL/6J×CBA) were used in all experiments. Suspensions of epididymal spermatozoa from sexually mature male mice were prepared for insemination of isolated oocytes. Oocytes were obtained from 28-day-old females superovulated with 10 IU PMSG and 10 IU hCG, injected intraperitoneally 48 h apart. Females were killed 16 h after hCG injection and both oviducts were immediately removed and placed in mineral oil.

In Vitro Fertilization with Cumulus-Oocyte Complexes

In vitro fertilization with cumulus intact oocytes was conducted with sperm dispersed from cauda epididymides placed for 5 min in 200 μl drops of fertilization medium under paraffin oil. The sperm suspension was diluted to a concentration of 10⁶ sperm/ml in a volume of 200 μl and then incubated for 120 min in a humidified tissue culture incubator (37° C., 5% CO₂ in air) to allow capacitation. In the experiments where anti-recmSLLP1 serum was tested, spermatozoa were incubated with varying concentrations of decomplemented (56° C., 30 min) immune or preimmune serum for the last 45 min of capacitation. In the experiments where recmSLLP1 was evaluated, spermatozoa were incubated under standard capacitating conditions.

Cumulus masses were placed in 135 μl drops of fertilization medium (one mass per drop) under paraffin oil and were incubated for 45 min with immune or preimmune serum or in presence or absence of recmSLLP1 prior to insemination. Fifteen μl of the sperm suspension (final concentration: 10⁵ sperm/ml) was then added to each cumulus mass drop. Thus, sera or recombinant protein was present in the incubation droplet during gamete interaction. Six hours following insemination oocytes were relocated in 100 μl drops of fertilization medium under mineral oil. Following overnight incubation, eggs were stained in Hoechst dye (10 μg/ml) for 10 min and washed 3 times in fertilization medium. The eggs were then placed in a 5 μl drop of fertilization medium between a microscope slide and an elevated coverslip, and visualized at 160× using light and fluorescence microscopy (Zeiss Axioplan). Two cells embryos were scored as fertilized, while one-celled oocytes were scored as unfertilized.

In Vitro Fertilization with Zona-Free Eggs

For the sperm-oolemma binding assay, two cauda epididymides were placed in 900 μl drops of fertilization medium under paraffin oil for the dense mass of spermatozoa to flow freely for ˜15 min and then diluted at 1×10⁶/ml for 3 h of capacitation. Cumulus oocyte complexes were placed in 200 μl drops of TYH medium under paraffin oil. Cumulus cells were removed by treating the oocytes for 3 min with 1 mg/ml hyaluronidase in TYH medium and then washed 8 times in 50 μl drops. Zona pellucidae were loosened by treating the oocytes with 10 μg/ml chymotrypsin in TYH media for 1 min and loosened zonae were removed by mechanical agitation using a pulled Pasteur pipette. The oocytes were then washed 10 times and allowed to recover from chymotrypsin treatment by incubating in TYH media for 3 h, following which, they were stained with 10 μg/ml Hoechst dye for 10 min, and then gently washed.

To test the effects of anti-recmSLLP1 antibodies, spermatozoa were incubated with varying concentrations of decomplemented immune or preimmune sera for the last 30 min of capacitation. Untreated oocytes were then added to the incubation drops containing the treated sperm with the final concentration being 2.5×10⁴ sperm/ml.

To compare the effects of recmSLLP1 with human and chicken c lysozymes, spermatozoa were first incubated under standard capacitating conditions. Oocytes were pre-incubated for 45 min before insemination with recmSLLP1 (0.1 to 200 μg/ml) or with hSLLP1 (25 μg/ml) or with chicken or human lysozyme (50 μg/ml, 100 μg/ml). Untreated capacitated sperm were then added to the incubation drops containing the treated eggs with a final sperm concentration of 2.5×10⁴/ml. Thus, in all the experiments performed the sera, the recombinant protein, or the lysozyme was present in the incubation droplet during gamete interaction. After 30 min of gamete co-incubation, oocytes were gently washed 5 times in TYH medium and placed between a microscope slide and an elevated coverslip and visualized at 160×. Binding to the oocyte was scored by counting the number of bound spermatozoa per oocyte using phase contrast. Fusion with the egg was scored by counting the number of decondensed sperm heads within each oocyte using fluorescence microscopy.

Indirect Immunofluorescence Studies of Mouse Spermatozoa and Oocytes

Localization of mSLLP1 on Fixed Spermatozoa

Cauda epididymal mouse spermatozoa were placed in 0.9 ml drop of phosphate-buffered saline without calcium (PBS; pH 7.4) (two epididymides per drop) and incubated at 37° C. in an atmosphere of 5% CO₂ for 5 min. To induce the acrosome reaction, spermatozoa were incubated in TYH media for 90 min to undergo capacitation (Visconti et al, 1995) and 5 μM calcium ionophore A23187 was added for another 15 min for the acrosome reaction to occur. Each drop was then collected, centrifuged for 10 min at 500 g and resuspended in PBS; this washing procedure was repeated three times. Smears of the final suspension of mouse sperm in PBS were air-dried on microscope slides at room temperature and fixed in 2% w/v paraformaldehyde in PBS for 10 min. After 6 washes in PBS, spermatozoa were incubated for 30 min at 37° C. with normal goat serum (NGS) (5% v/v in PBS) and then incubated for 1 h with anti-recmSLLP1 sera (1:25). The slides were washed 3 times in PBS and spermatozoa were incubated for 1 hour at 37° C. with Texas red-conjugated polyclonal antibody from donkey (1:200, Jackson Laboratories). Slides were then washed, incubated for 30 min at room temperature with Peanut agglutinin lectin (PNA) (1:50) (Molecular Probes) conjugated with FITC, washed, mounted in Slowfade® (Molecular Probes, Eugene, Oreg.), and visualized under a Zeiss Standard 18 ultraviolet microscope. Images were captured by using MrGrab (Carl Zeiss Vision GmbH, Germany).

Egg labeling: Metaphase II eggs were obtained as previously described (Coonrod et al, 1999) and incubated with 5% NGS/media for 30 min. Oocytes were washed five times in TYH medium and incubated with 100 μg/ml recmSLLP1 or 100 μg/ml lysozymes for 45 min at 37° C. and 5% CO₂. Oocytes were washed five times and incubated with guinea pig anti-recmSLLP1 polyclonal antibody (1:50), sheep anti-human lysozyme (1:25) or rabbit anti-chicken lysozyme (1:400) in 5% NGS/media for 1 h at 37° C. and 5% CO₂. Oocytes were washed five times and incubated with donkey anti-guinea pig/Texas Red antibody (1:200) or goat anti-guinea pig/FITC antisera 1:200), donkey anti-sheep and goat anti-rabbit FITC-labeled secondary antibody (1:200) (Jackson ImmunoResearch), respectively in 5% NGS/media for 1 h at room temperature at 37° C. and 5% CO₂. Oocytes were washed and mounted in media onto glass slides and visualized under a Zeiss Standard 18 ultraviolet microscope. Images were captured by using MrGrab 1.0 (Carl Zeiss Vision GmbH, Germany).

Scanning Confocal Microscopy

Metaphase II eggs employed for immunofluorescence studies (above) were utilized for scanning confocal microscopy. The stained eggs were washed three times in PBS containing 1% BSA (PBS/BSA) and then fixed in 4% paraformaldehyde in PBS-polyvinylalcohol (PVA) for 20 min at room temperature. Following fixation, eggs were washed 5 times in PBS/BSA and then permeabilized with 0.5% Triton X-100 in PBS for 20 min at room temperature. Eggs were then washed five times in PBS/BSA and placed in 0.4 mg/ml RNase in PBS/BSA for 30 min and then stained with 20 nM Sytox (Molecular Probes) for 10 min. Eggs were then extensively washed, placed in slow fade (Molecular Probes) equilibration media for approximately 1 min and then mounted on slides in slow fade mounting media. Images were obtained on a Zeiss 410 Axiovert 100 Microsystems LSM confocal microscope. For each panel, attenuation, contrast, brightness and pinhole aperture remained constant. For each panel, four seconds scans were averaged four times per line using a 63× oil lens equipped with a zoom factor of two.

Sperm Labeling During Binding to Metaphase II Eggs

Zona-free eggs inseminated with capacitated spermatozoa from the in vitro fertilization studies above were fixed with 2% paraformaldehyde for 10 min at room temperature. Gametes were washed in PBS-BSA, incubated with 5% NGS/PBS-BSA for 30 min at 37° C. and then incubated for 1 h with anti-recmSLLP1 sera (1:25). The slides were washed 3 times in PBS and gametes were incubated for 1 h at 37° C. with donkey anti-guinea pig Texas red-conjugated polyclonal antibody (1:200, Jackson Laboratories). Gametes were then washed five times in PBS/BSA, placed in 0.4 mg/ml RNase in PBS/BSA for 30 min, and then stained with 20 nM Sytox (Molecular Probes) for 10 min for nuclear staining. Gametes were extensively washed, placed in slow fade (Molecular Probes) equilibration media for approximately 1 min and then mounted on slides in slow fade mounting media. Images were obtained on a Zeiss 410 Axiovert 100 microsystems LSM confocal microscope as described above.

Statistical Analysis

All in vitro assays were repeated at least three or more times. Experimental and control group values were reported as means±standard error of mean. Groups were compared using the matched-pairs t-test assuming equal variances and differences were reported at P≦0.05 as the level of significance (Bowers, D. Medical Statistics from Scratch. John Wiley & Sons; West Sussex, UK, 2002, pp. 129-132).

Results

Mouse SLLP1 is the True Orthologue of hSLLP1 and Shares Similar Characteristics to c Lysozymes

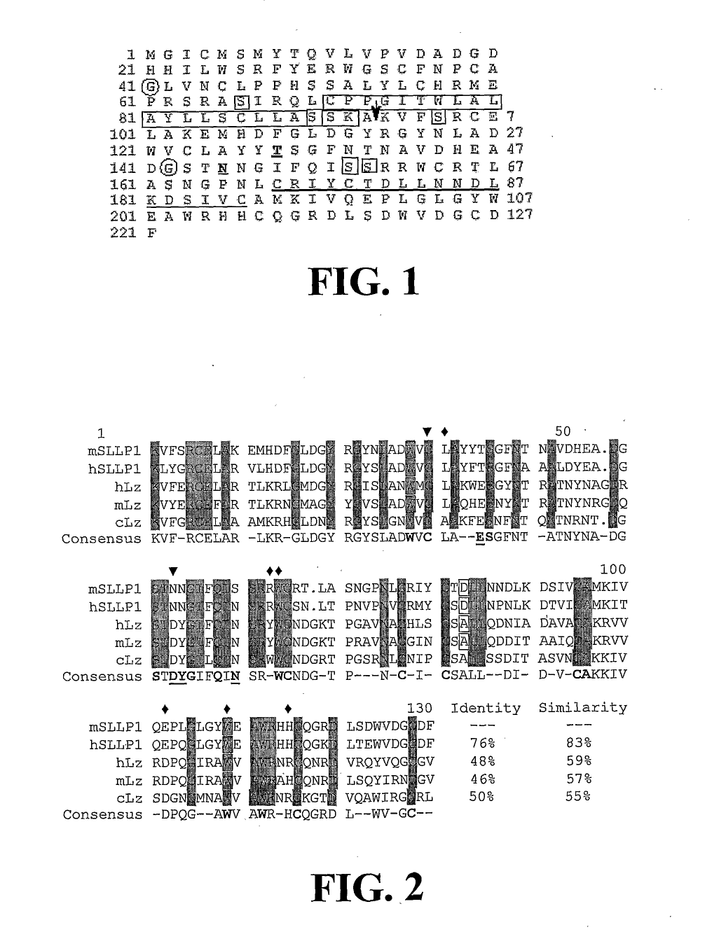

The complete deduced amino acid sequence of mSLLP1 is shown in FIG. 1 (predicted mol. wt. 25 kDa, pI-6.2). The N terminus of mSLLP1 contains a predicted transmembrane domain followed immediately by a potential protease cleavage site between alanine 93 and lysine 94 linkage. Comparison of the full-length hSLLP1 and mSLLP1 sequences using the Accelrys Gap (Seq/Web version 2) algorithm found that mSLLP1 is 64.2% similar and 58.8% identical to the hSLLP1. The mSLLP1 processed form, starting after the protease cleavage site (128 amino acids), shares 82.8% similarity and 75.8% identity to that of hSLLP1. The deduced mSLLP1 sequence contains three putative myristoylation sites (G2, G41 and G142), potential phosphorylation sites for casein kinase II (S97) and protein kinase C(S66, S90, S152, and S153) and a signature sequence for the alpha-lactalbumin/lysozyme C family. The predicted molecular weight (14.6 kDa) and pI (5.2) of mature mSLLP1 are identical to hSLLP1.

In addition, a Blast search of the NCBI GenBank database and a multiple sequence alignment of selected mature c lysozymes revealed that mature mSLLP1 is 46%, 48% and 50% identical to mouse, human and chicken lysozymes, respectively (FIG. 2). Forty-one residues in mSLLP1 are identical to the three conventional lysozymes. Among the 20 invariant residues of c lysozymes (Prager, E. M., and Jolles, P., Animal lysozymes c and g: an overview. In: Jolles, P. (Ed.), Lysozymes: Model Enzymes in Biochemistry and Biology. Birkhauser Verlag, Basel, 1996, pp. 9-31), 16 were found to be conserved in mSLLP1. Interestingly, the essential catalytic residues (E35 and D52) of chicken lysozyme were replaced with T35 and N52 in mSLLP1 as well as hSLLP1 (Prager, E. M., and Jolles, P., Animal lysozymes c and g: an overview. In: Jolles, P. (Ed.), Lysozymes: Model Enzymes in Biochemistry and Biology. Birkhauser Verlag, Basel, 1996, pp. 9-31). Among the six potential substrate-binding residues of c lysozymes, five were conserved in both mSLLP1 and hSLLP1 (Kumagai, I., Sunada, F., Takeda, S., and Miura, K., 1992. Redesign of the substrate-binding site of hen egg white lysozyme based on the molecular evolution of C-type lysozymes. J. Biol. Chem. 267, 4608-4612).

The mouse SLLP1 gene, Spaca3, is a six exon gene located on chromosome 11 at locus B5 where it is flanked by the gene for amiloride-sensitive cation channel 1, Accn1, and an unknown protein belonging to the myosin family. This locus in the mouse is considered syntenic with 17q12 where the human SLLP1 gene, SPACA3, is also flanked by ACCN1 and the myosin gene MYO1D. Furthermore, the intron positions of mature mouse and human SLLP1 precisely match with that for human and mouse lysozymes interrupting codons for Trp, Asp/Ala and Trp (FIG. 2), suggesting a possible origin of these genes from a common ancestor.

Expression of mSLLP1 and Specificity of the Antibody

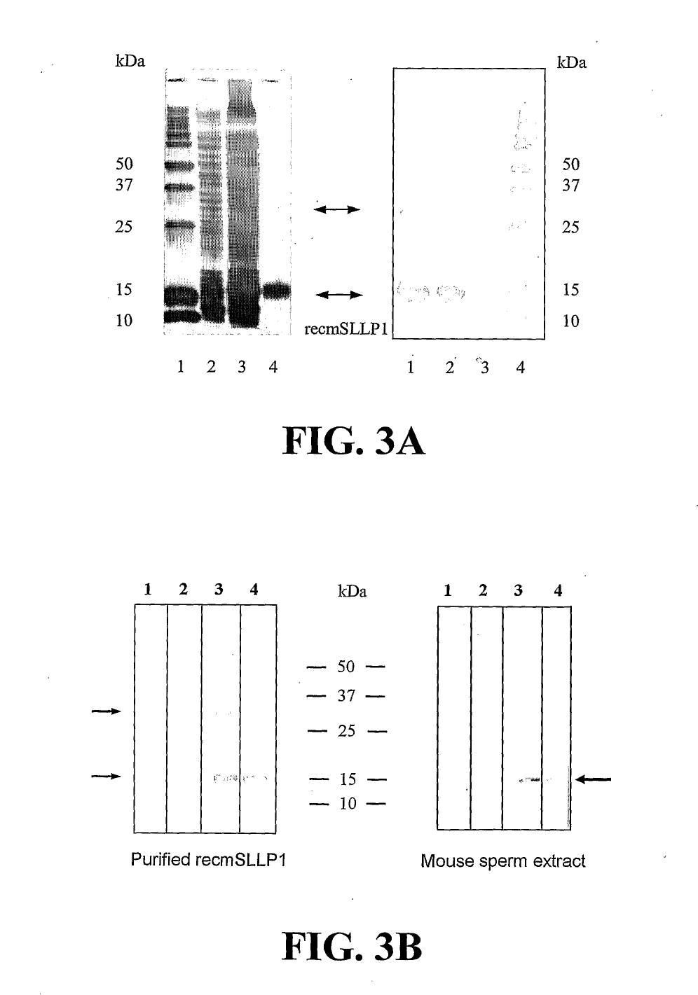

A cDNA sequence encoding the mature mSLLP1 from residue 94 to 221 (beginning after the putative protease cleavage site), and including a six-histidine C terminal tag was expressed in E. coli and a recombinant protein of about 15 kDa was obtained after Ni⁺⁺-affinity purification. To evaluate the relative purity of the recmSLLP1 preparation, an aliquot of the purified protein was separated by 1-D electrophoresis and the gel was silver stained and blotted to probe with anti-his antibody. A prominent band of about 15 kDa and a much fainter putative dimer at about 30 kDa (FIG. 3A) were noted. These results indicated that the recmSLLP1 preparation used for this study was highly purified.

The specificity of the antibody generated in guinea pigs against recmSLLP1 was examined by western blotting against both the recombinant immunogen and mouse sperm proteins. The immune sera recognized the 15 kDa recombinant SLLP1 as well as the putative 30 kDa dimer found in the recombinant preparation, while the preimmune serum as well as the serum from guinea pigs injected with adjuvant alone showed no immunoreactivity with recSLLP1 (FIG. 3B). In mouse sperm extracts the immune serum reacted only with a about 15 kDa band while the serum from guinea pigs injected with adjuvant alone as well as preimmune sera showed no reactivity (FIG. 3B). These results indicated that a specific immunoreagent had been generated to the recmSLLP1 that gave a single band on sperm protein extracts. Finding only a ˜15 kDa form of mSLLP1 in sperm protein extracts demonstrated that full length mSLLP1, predicted to run at ˜25 kDa, was not detectable in sperm. Further mSLLP1 dimerization does not occur in sperm although a small amount does occur during E. coli expression. The observations also indicated that E. coli expressed mSLLP1 after affinity purification contained sufficient numbers of immunogenic epitopes to generate antibodies cross reactive with the native mSLLP1.

Mouse SLLP1 is Associated with Sperm Acrosome and Retained after Acrosome Reaction

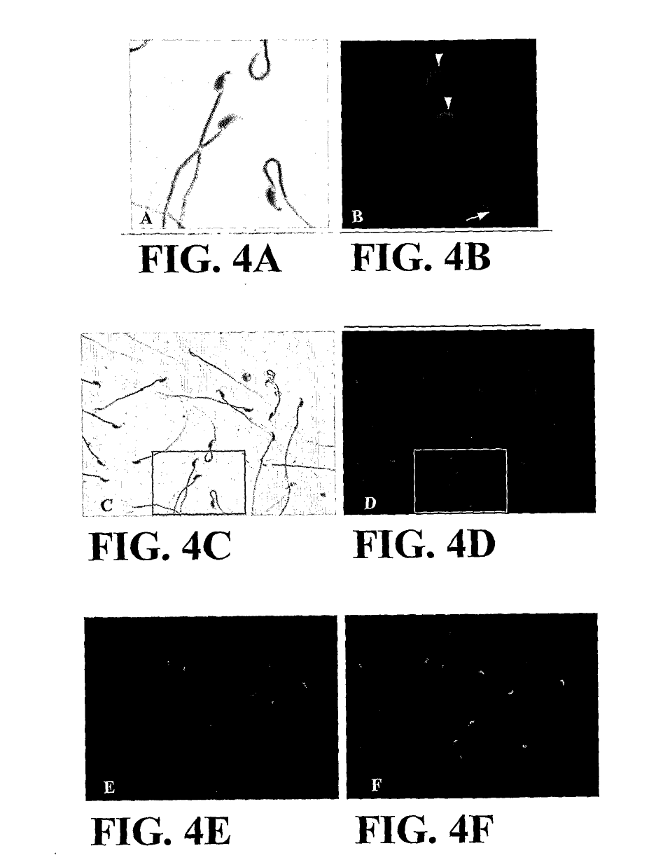

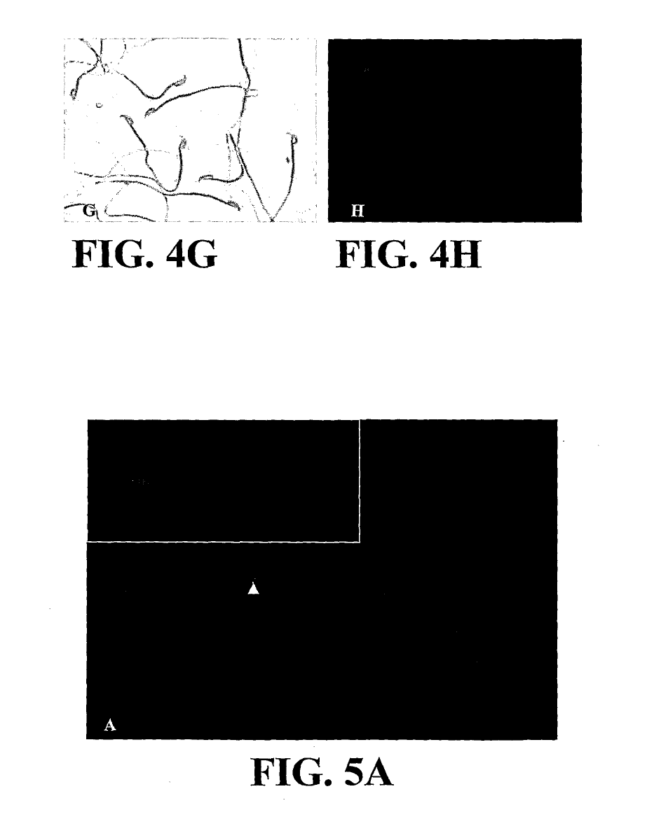

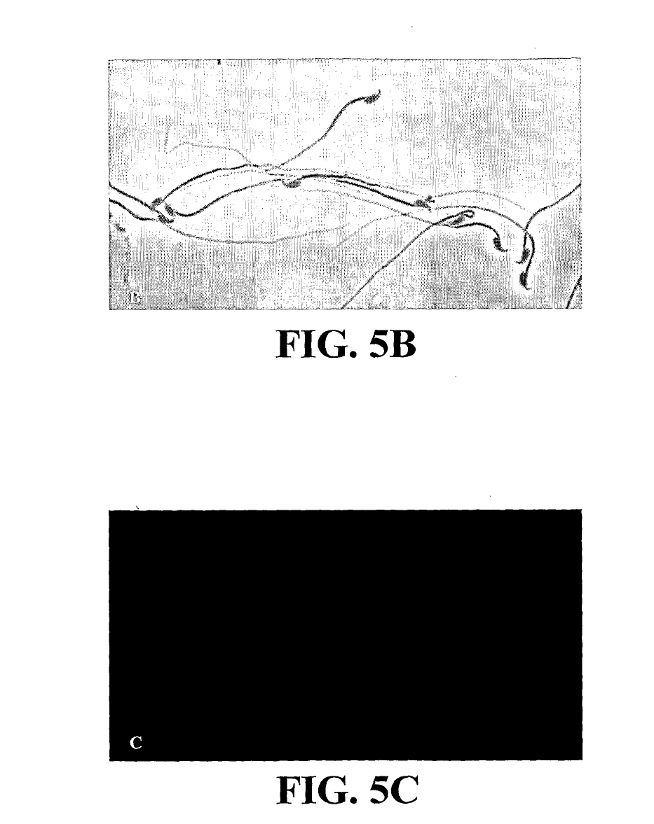

Indirect immunofluorescence of fixed mouse spermatozoa localized mSLLP1 to the anterior acrosome in 70.0% of non-capacitated caudal spermatozoa (FIG. 4, Table 1; all tables are attached after the Abstract). However, 20.3% of non-capacitated spermatozoa showed an equatorial segment distribution of mSLLP1 and 4.5% possessed no staining (Table 1). Seventy-one percent of acrosome-reacted sperm, determined by the lack of fluorescence in the acrosome by PNA lectin staining, displayed only equatorial segment reactivity with anti-recmSLLP1 serum (FIG. 5). However, 13.9% of acrosome-reacted spermatozoa retained an anterior acrosome staining pattern while 9.3% did not stain (Table 1). The disappearance of mSLLP1 staining in the anterior acrosome appeared to correspond with the appearance of staining in the equatorial segment following capacitation and the acrosome reaction.

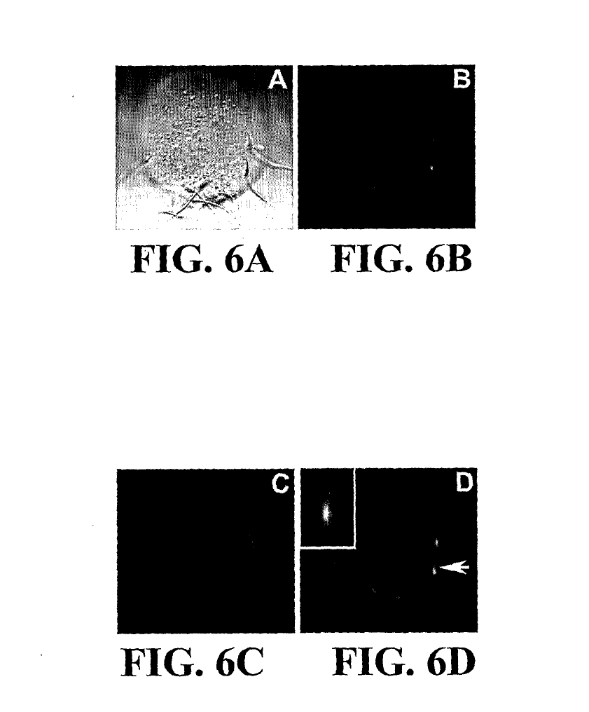

Proteins from acrosome reacted sperm were analyzed by Western analysis (Table 1 insert) using anti-recmSLLP1 serum. Ionophore treated, acrosome reacted sperm showed a ˜14 kD mSLLP1 band with very subtle decrease in migration compared to proteins extracted from capacitated sperm. The slower migration of the ˜14 kD band is possibly due to changes in phosphorylation/dephosphorylation of mSLLP1 during the acrosome reaction, a process known to be regulated by protein phosphorylation (Furuya et al, 1992). The presence of mSLLP1 in ionophore treated as well as capacitated sperm confirmed that the observed equatorial immunofluorescent staining is a specific SLLP1 pattern. Importantly, confocal analysis showed retention of mSLLP1 in all capacitated mouse sperm tightly bound to mouse eggs (FIG. 6), emphasizing the concept that this protein may be involved in sperm-oolemma binding.

RecmSLLP1 and Anti-RecmSLLP1 Serum Inhibit Fertilization of Mouse Cumulus Intact Eggs

To determine the role of mSLLP1 during fertilization, both spermatozoa and cumulus intact oocytes were pre-incubated with anti-recmSLLP1 serum or preimmune serum for 45 min prior to insemination. Fertilization was conducted in the presence of the antibody and six hours later the eggs were relocated in 100 μl drops of fertilization medium and incubated overnight. In the groups treated with the immune sera at 1:10 or 1:50 dilutions, the percentage of two cells embryos was significantly (P≦0.05) reduced (61% and 17% inhibition respectively; Table 2A) from the values observed with respective preimmune serum. However, a significant effect on cumulus intact eggs was not observed at a 1:100 dilution.

Cumulus intact oocytes were also incubated with two concentrations of recmSLLP1, which remained in the culture medium during the fertilization process. Treatment of the cumulus-oocyte complexes with 200 μg/ml recmSLLP1 significantly reduced the fertilization rate from 45% in the control group to 12% in the recmSLLP1 treated group (73% inhibition, P≦0.05), whereas no significant difference was observed on the percentage of fertilization between the control group and the group treated with 50 μg/ml recombinant protein, although a reduction was noted (Table 2B). Taken together, these results suggested that mSLLP1 plays a role in fertilization.

Mouse SLLP1 has a Role in Sperm-Egg Binding

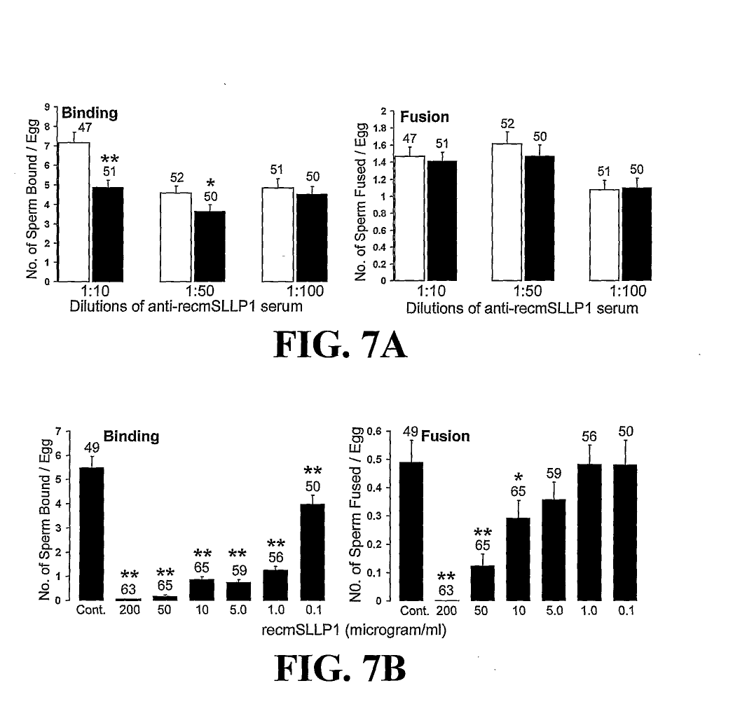

Inhibition of fertilization by recmSLLP1 protein as well as antibodies to recmSLLP1 using cumulus-egg complexes prompted a dose-ranging study of the effect of antibody to SLLP1 and recmSLLP1 on gamete binding and fusion to determine the stage in the fertilization cascade at which mSLLP1 exerted its effects. Therefore, we tested whether anti-recmSLLP1 serum (FIG. 7A) and recmSLLP1 protein (FIG. 7B) would block capacitated mouse sperm-egg binding, fusion, or both to zona-free mouse eggs. Statistically significant inhibition of binding but not fusion was observed when both gametes were co-incubated in the presence of 1:10 and 1:50 dilutions of anti-recmSLLP1 immune sera compared to preimmune sera, whereas the 1:100 dilutions were not significantly different (FIG. 7A).

The most striking effect was observed when zona-free mouse eggs were incubated with different concentrations of recmSLLP1 (0.1-200 μg/ml) and then inseminated with untreated capacitated mouse spermatozoa. The incubation of oocytes with recmSLLP1 produced a concentration-dependent decrease in the number of spermatozoa bound to or fused to the egg, with a significant effect observed at as low as 0.1 μg/ml and 100% inhibition of both binding and fusion at 200 μg/ml (12.5 μM; FIG. 7B). Zona-free mouse oocytes incubated in the absence of recmSLLP1 (buffer only) was used as control. Importantly, no difference was observed in the percentages of motile spermatozoa compared to the control suggesting that anti-recmSLLP1 or recmSLLP1 protein did not affect sperm motility but oolemma binding and subsequent fusion. Taken together, these results support the participation of mSLLP1 in the binding event at the mouse egg surface prior to fertilization.

Mouse SLLP1 has Complementary Binding Sites on Unfertilized and Fertilized Oocytes

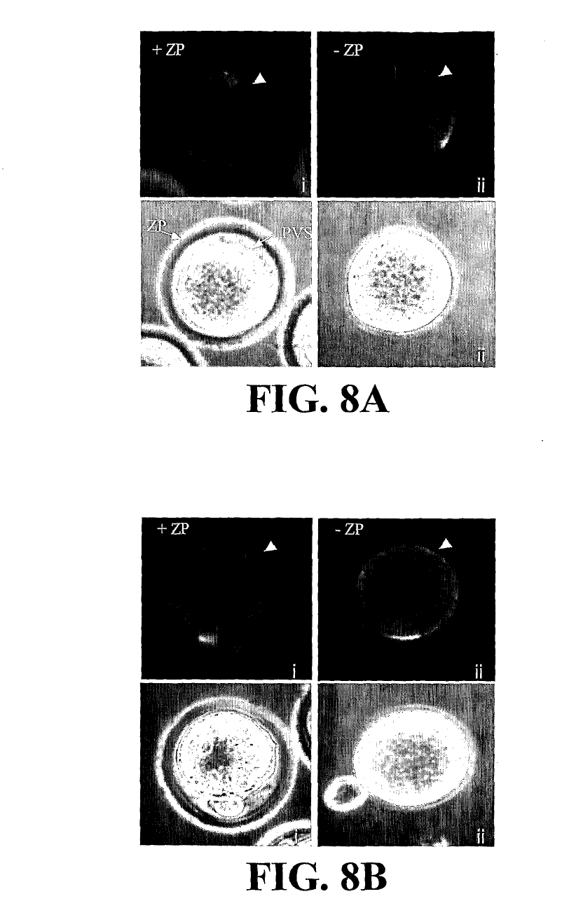

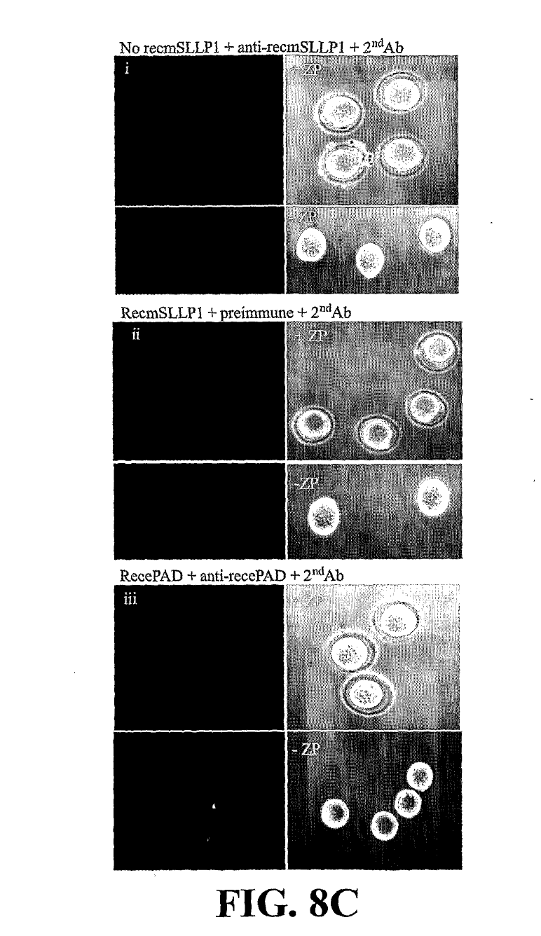

To study the possible localization of mSLLP1-binding sites on the egg surface, unfertilized oocytes along with in vitro fertilized oocytes at the pronuclear stage, were incubated with purified recombinant mSLLP1 for 45 min, washed, and then exposed to anti-recmSLLP1. Unfertilized oocytes exhibited fluorescent labeling within the perivitelline space and over much of the oocyte surface (FIG. 8Ai and ii; upper panels, white arrowheads). However, an area devoid of fluorescence was consistently detected. Hoechst staining revealed that this negative area was always associated with the area of the oocyte plasma membrane overlying the metaphase plate (FIG. 8Aii, lower panel). Thus, mSLLP1-binding sites were restricted to the fusogenic region of the egg, consistent with a role for mSLLP1 interaction in sperm-egg binding. Interestingly, oocytes, with or without zona pellucida, that had been fertilized in vitro and treated with recmSLLP1, exhibited intense, patchy immunofluorescent domains over the entire egg surface (FIG. 8B, white arrowheads), indicating that SLLP1 binding sites remain associated with egg surface domains after fertilization. Controls included oocytes not exposed to recmSLLP1 and then exposed to anti-recmSLLP1 (FIG. 8Ci), oocytes incubated with recmSLLP1 and then exposed to preimmune sera (FIG. 8Cii), and oocytes incubated with recePAD (an egg cytoplasmic protein; Wright et al., 2003, ePAD, an oocyte and early embryo-abundant peptidylarginine deiminase-like protein that localizes to egg cytoplasmic sheets. Dev. Biol. 256, 73-88) and then incubated with the respective specific antibody (FIG. 8C iii). None of these three controls showed egg surface fluorescence.

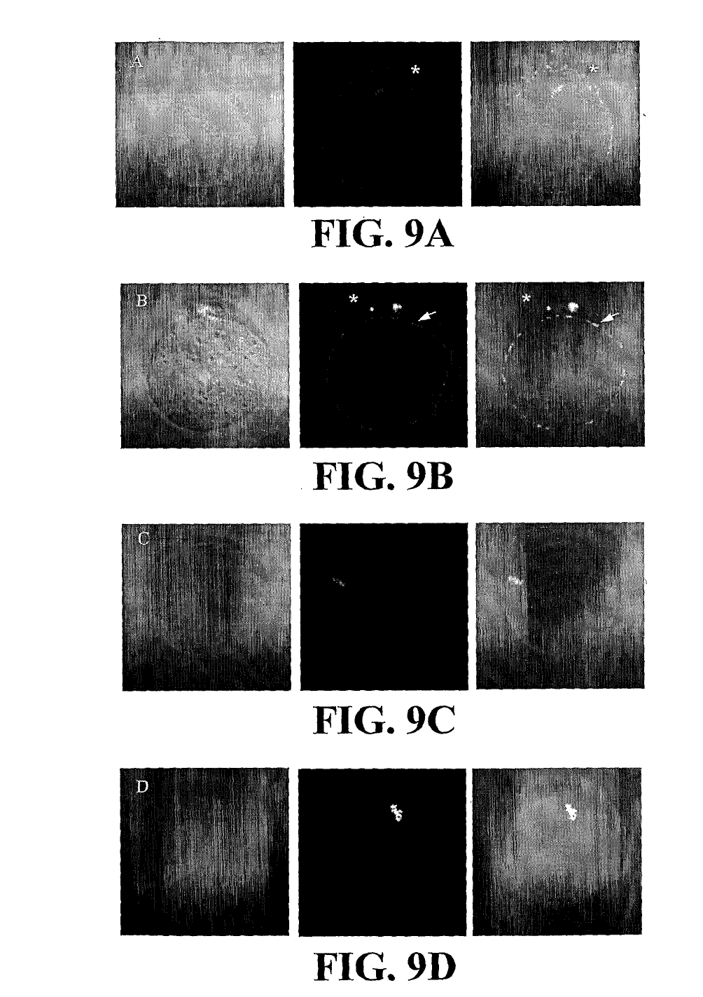

Confocal analyses were then employed to refine the localization of mSLLP1 binding sites in the egg after treatment with recombinant SLLP1. Optical sections of zona intact unfertilized oocytes showed that mSLLP1-binding sites were localized predominantly to the perivitelline space (FIG. 9A). In contrast, fluorescence was virtually undetected in the perivitelline space of fertilized eggs while an intense signal for SLLP1 was evident on the oolemma in distinct patches (FIG. 9B). A weak fluorescent labeling was also observed on zona pellucidae of both unfertilized and fertilized eggs.

C Lysozymes do not Block Gamete Binding or Fusion to the Mouse Egg

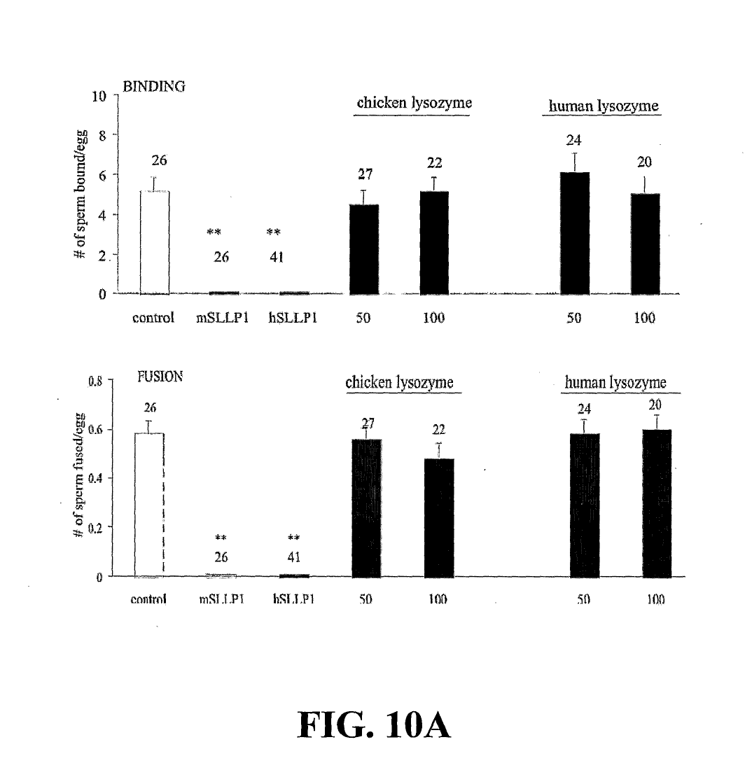

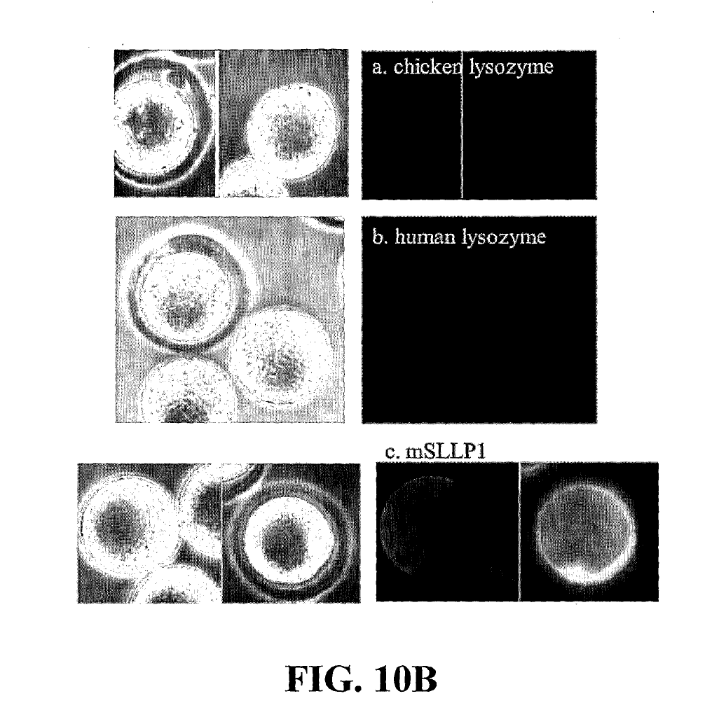

Mouse and human SLLP1 are lysozyme-like proteins that share several characteristics of the c lysozyme family. Mouse SLLP1 is 48% identical to human and 50% identical to chicken lysozyme, 46% identical to conventional mouse lysozyme, while human SLLP1 is 52% identical to human lysozyme and 48% identical to chicken lysozyme. To determine whether conventional c lysozymes have an inhibitory effect similar to SLLP1 on sperm-egg binding and fusion, human and chicken lysozyme were incubated with mouse oocytes at concentrations shown previously to exert maximal effects for SLLP1s (FIG. 10). Although 50 μg of mouse SLLP1 and 25 μg of human SLLP1 maximally inhibited sperm-egg binding and fusion, a similar effect was not observed even at 50 or 100 μg/ml of the conventional c lysozymes (FIG. 10A). Similarly, mouse oocytes did not show any fluorescence when incubated with human or chicken c lysozymes and their respective antibodies (FIG. 10B), indicating a lack of oolemmal receptors for these c lysozymes.

Table 1: Incidence of SLLP1 staining patterns in populations of acrosome intact and acrosome reacted mouse spermatozoa. In acrosome intact sperm, defined by positive PNA staining, mSLLP1 localized mainly to the anterior acrosome (70.0%), secondarily to the equatorial segment (20.3%), whereas 4.5% of the cells displayed no staining. In capacitated and ionophore-induced acrosome reacted sperm this pattern was reversed, mSLLP1 was localized to the anterior acrosome in only 13.9% of spermatozoa, whereas mSLLP1 localized to the equatorial region in 70.9% of the cells. The percentage of acrosome reacted (AR) sperm in the population is addressed in the last column.

The insert shows Western analysis of the capacitated (CP) and acrosome reacted (AR) mouse cauda epididymal sperm probed with preimmune (Pi) and immune (Im) serum. The acrosome reacted sperm clearly demonstrated the retention of some mSLLP1, a 14 kD band (arrow head) in the acrosome reacted population.

TABLE 1

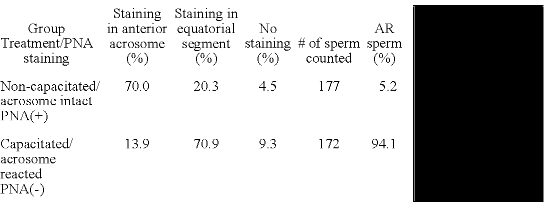

Table 2 (comprising Tables 2A and 2B): Effect of SLLP1 anti-serum and recmSLLP1 on mouse in vitro fertilization using cumulus intact oocytes. In all cases, the sera or the recombinant proteins were present during fertilization. Two cells embryos were scored as fertilized after 24 h. (*) P≦0.05. (A) Decomplemented preimmune (PI) or anti-recmSLLP1 immune (I) sera were added to both gametes 45 min prior to insemination. Statistically significant inhibition was seen at 1:10 and 1:50 dilutions. (B) RecmSLLP1 was added to the oocytes 45 min prior to insemination with untreated capacitated mouse spermatozoa. Significant inhibition was noted at 200 μg/ml of mSLLP1. Control oocytes were pre-incubated with PBS containing no recmSLLP1.

TABLE 2A

| | | | # of | # of non | |
|---|---|---|---|---|---|
| | # of | total # | 2 cells | fertilized | % |
| Treatment | experiments | of eggs | embryo | eggs | Fertilization |
| PI 1:100 | 3 | 20 | 15 | 5 | 75 |
| I 1:100 | 3 | 20 | 14 | 6 | 70 |
| PI 1:50 | 3 | 38 | 27 | 11 | 71 |
| I 1:50 | 3 | 46 | 27 | 19 | 59* |
| PI 1:10 | 6 | 64 | 43 | 21 | 67 |
| I 1:10 | 6 | 80 | 21 | 59 | 26* |

TABLE 2B

| | | | # of | # of non | |
|---|---|---|---|---|---|
| | # of | total # | 2 cells | fertilized | % |
| Treatment | experiments | of eggs | embryo | eggs | Fertilization |
| Control | 3 | 20 | 11 | 9 | 55 |
| recmSLLP1 | 3 | 52 | 25 | 27 | 48 |
| 50 μg/ml | | | | | |
| Control | 3 | 29 | 13 | 16 | 45 |
| recmSLLP1 | 3 | 64 | 8 | 56 | 12* |
| 200 μg/ml | | | | | |

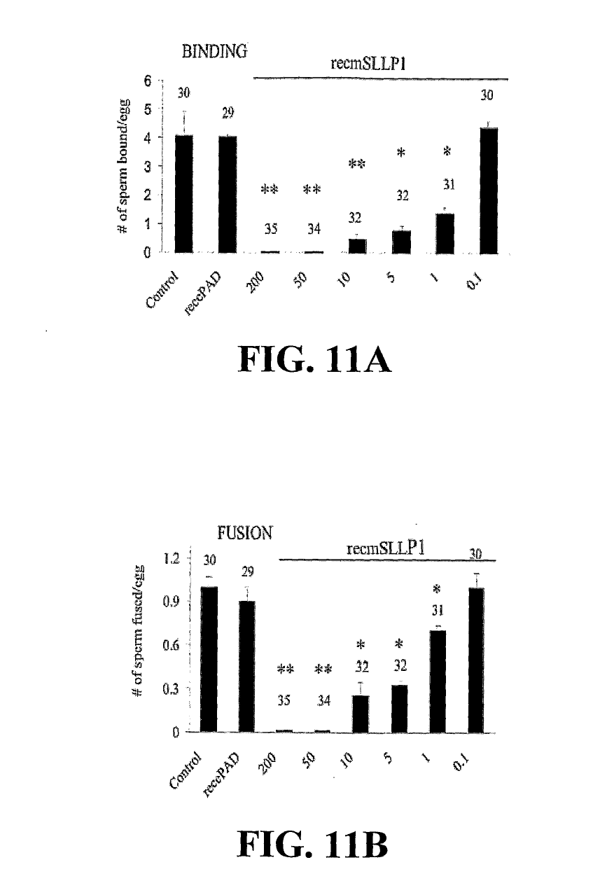

Additionally, it can be seen in the two graphs of FIG. 11 that recombinant mouse SLLP1 can essentially completely inhibit sperm-egg binding, as measured by number of sperm bound/egg (left panel), as well as sperm-egg fusion (right panel), relative to the controls, including comparison to a recombinant egg protein, ePAD.

### Example 2

### Sperm Protein SLLP2

### Role in Sperm Binding with Egg and Use to Identify Egg Receptors for Sperm

Human SLLP1 and SLLP2 nucleic acids and proteins were previously identified and sequenced by the inventors (see U.S. patent application Ser. No. 10/181,611, filed Jul. 18, 2002, the entirety of which is incorporated herein by reference). These proteins were further characterized, and other members of the family were identified by the present inventors (see U.S. patent application Ser. No. 10/542,038 filed Jul. 13, 2005, the entirety of which is incorporated herein by reference). Both SLLP proteins are sperm specific in their expression.

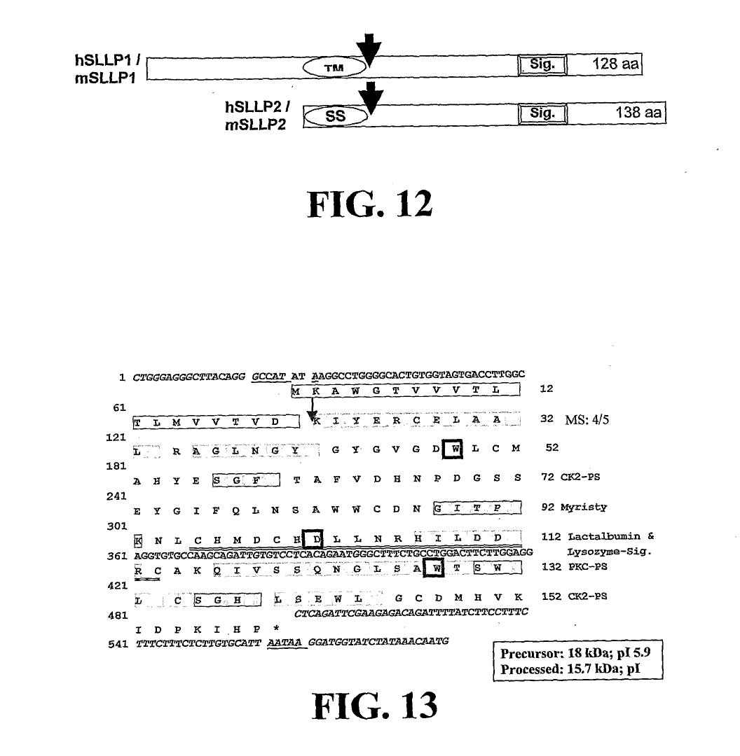

A schematic comparison of human and mouse SLLP1 and 2 is provided in FIG. 12. Additionally, the human SLLP2 cDNA and deduced amino acid sequences were determined and are provided in FIG. 13. The precursor SLLP2 is about 18 kDA, with a pI of 5.9 and the processed form is 15.7 kDA and the pI is 5.9.

Human SLLP1 and SLLP2 each contain a signal peptide. The initial SLLP1 polypeptide is synthesized as a 215 amino acid polypeptide having a MW of 23.4 kDa and a pI of 8.0. The mature SLLP1 peptide is 128 amino acids and has a MW of about 14.6 kDa and pI of 5.0. The initial SLLP2 polypeptide is synthesized as a 159 amino acid polypeptide having a MW of 17.9 kDa and a pI of 5.9. The mature SLLP2 peptide is 138 amino acids and has a MW of about 15.7 kDa and pI of 5.9.

Human SLLP1 and SLLP2 have 48.8% sequence identity between one another and have 52% and 44% amino-acid sequence identity with the one known mature human lysozyme C, respectively, and 44% and 43% amino-acid sequence identity with the predicted lysozyme homologue on chromosome 17q11.2. SLLP1 is most closely related to human lysozyme (52% sequence identity), whereas SLLP2 is most closely related to chicken lysozyme (51% sequence identity).

The gene encoding SLLP1 is located on Chromosome 17 and is 6012 bp in length. The SLLP1 gene contains 5 exons (109, 309, 159, 79 and 164 bp, respectively) and 4 introns (3436, 1125, 443 and 188 bp, respectively). The gene encoding SLLP2 is located on Chromosome Xp11.1 and is 1950 bp in length. The SLLP2 gene contains 4 exons (169, 159, 79 and 181 bp, respectively) and 3 introns (428, 830, and 104 bp, respectively). Interestingly, exons 3 and 4 of SLLP1 have a sequence identity with exons 2 and 3 of SLLP2 greater than the overall sequence identity between the two complete proteins (i.e. greater than 48.8%) and exons 3 and 4 of SLLP1 are identical in size to exons 2 and 3 of SLLP2, respectively.

Mature Mouse SLLP2 cDNA Sequence (SEQ ID NO:15):
AAGATTTATGAACGCTGTGAGCTGGCAAAGAAGCTGGAGGAGGCTGGCCT
CGATGGCTTCAAAGGCTATACTGTTGGAGACTGGCTGTGTGTGGCACACT
ATGAGAGTGGCTTTGACACCTCTTTTGTGGACCACAATCCAGATGGCAGC
AGTGAATATGGCATTTTCCAGCTGAACTCTGCCTGGTGGTGTAACAATGG
CATCACACCCACTCAGAACCTCTGCAACATCGATTGTAATGACCTGCTCA
ACCGCCATATTCTGGATGATATCATATGTGCCAAGAGGGTTGCATCCTCA
CATAAGAGTATGAAGGCCTGGGATTCCTGGACCCAGCACTGTGCCGGTCA
TGATTTATCAGAATGGCTAAAGGGGTGTTCTGTGCGTCTGAAAACTGACT
CAAGCTATAATAACTGG
Mature Mouse SLLP2 ammo acid Sequence
(SEQ ID NO:16):
KIYERCELAKKLEEAGLDGFKGYTVGDWLCVAHYESGFDTSFVDHNPDGS
SEYGIFQLNSAWWCNNGITPTQNLCNIDCNDLLNRLDDIICAKRVASSHK
SMKAWDSWTQHCAGLIDLSEWLKGCSVRLKTDSSYNNW
Human SLLP2 cDNA sequence (SEQ ID NO:17):
CTGGGAGGGCTTACAGGTGCCATAATGAAGGCCTGGGGCACTGTGGTAGT
GACCTTGGCCACGCTGATGGTTGTCACTGTGGATGCCAAGATCTATGAAC
GCTGCGAGCTGGCGGCAAGACTGGAGAGAGCAGGGCTGAACGGCTACAAG
GGCTACGGCGTTGGAGACTGGCTGTGCATGGCTCATTATGAGAGTGGCTT
TGACACCGCCTTCGTGGACCACAATCCTGATGGCAGCAGTGAATATGGCA
TTTTCCAACTGAATTCTGCCTGGTGGTGTGACAATGGCATTACACCCACC
AAGAACCTCTGCCACATGGATTGTCATGACCTGCTCAATCGCCATATTCT
GGATGACATCAGGTGTGCCAAGCAGATTGTGTCCTCACAGAATGGGCTTT
CTGCCTGGACTTCTTGGAGGCTACACTGTTCTGGCCATGATTTATCTGAA
TGGCTCAAGGGGTGTGATATGCATGTGAAAATTGATCCAAAAATTCATCC
ATGACTCAGATTCGAAGAGACAGATTTTATCTTCCTTTCATTTCTTTCTC
TTGTGCATTTAATAAAGGATGGTATCTATAAACAATGC
Human SLLP2 Amino acid sequence (SEQ ID NO:18):
MKAWGTVVVTLATLMVYTVDAKIYERCELAARLERAGLNGYKGYGVGDWL
CMAHYESGFDTAFVDHNPDGSSEYGIFQLNSAWWCDNGITPTKINLCHMD
CHDLLNRHILDDIIRCAKQTVSSQNGLSAWTSWRLHCSGHDLSEWLKGCD
MHVKIDPKIHP

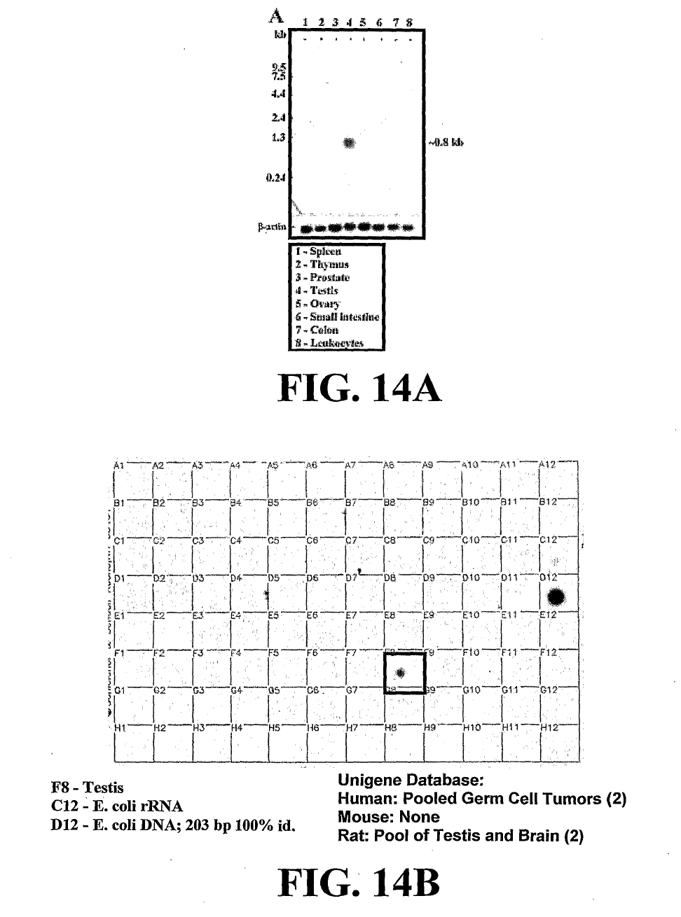

Proof of the sperm specificity of expression of SLLP2 is provided in FIG. 14, as indicated by northern blot analyses of spleen, thymus, prostate, testis (lane 4), ovary, small intestine, colon, and leukocytes (FIG. 14A). See also the grid of FIG. 14B, where the square labeled F8 represents testis and the square D12 represents the gene as cloned and expressed in bacteria.

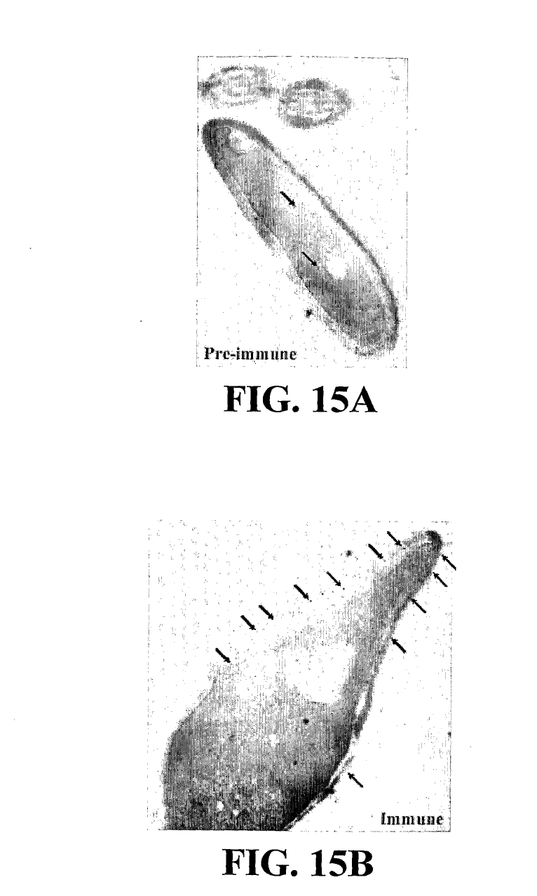

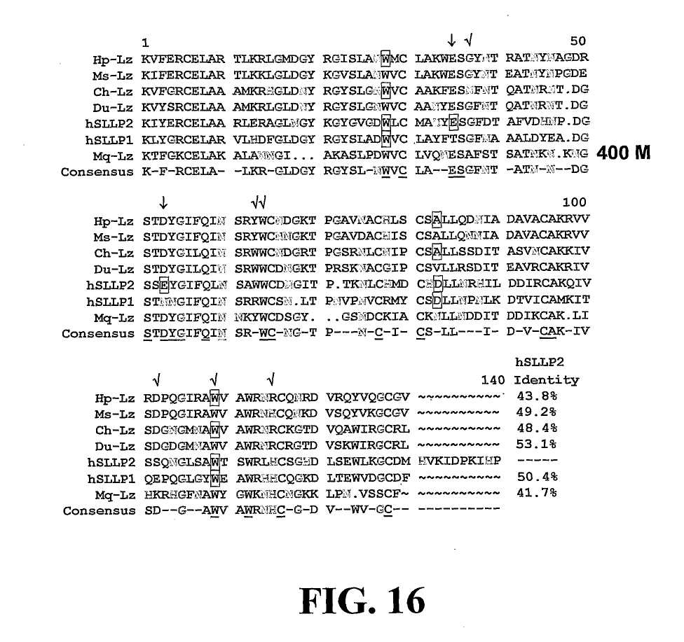

Recombinant human SLLP2 (“rechSLLP2”) was prepared and expressed in E. coli. Antibodies were prepared against the recombinant SLLP2. It was then shown by immuno-electron microscopy that human SLLP2 protein is localized to the sperm acrosomal region in human sperm (see FIG. 15). The alignment of human SLLP2 protein with it homologues is shown in FIG. 16. Soluble human SLLP2 was also isolated and purified, as was mouse SLLP2.

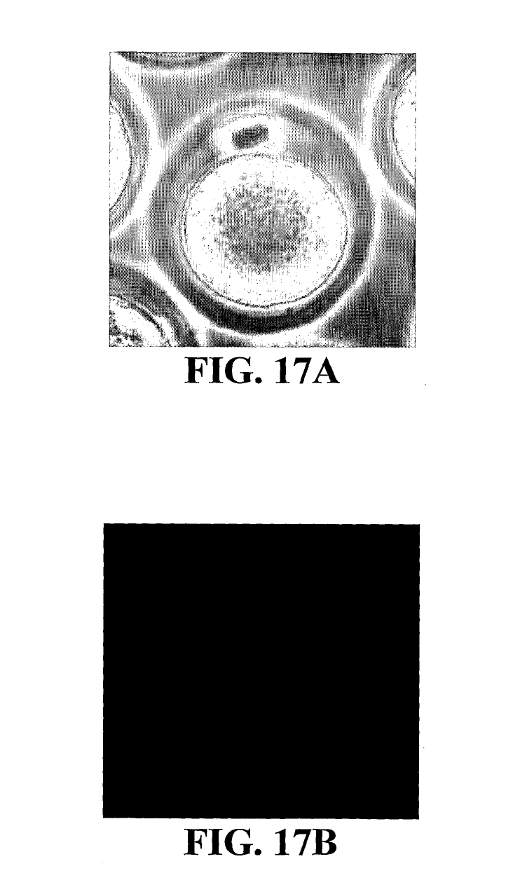

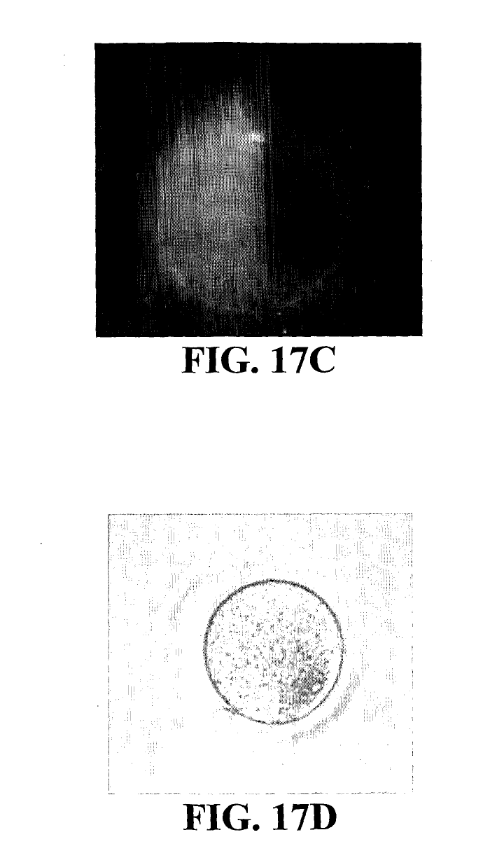

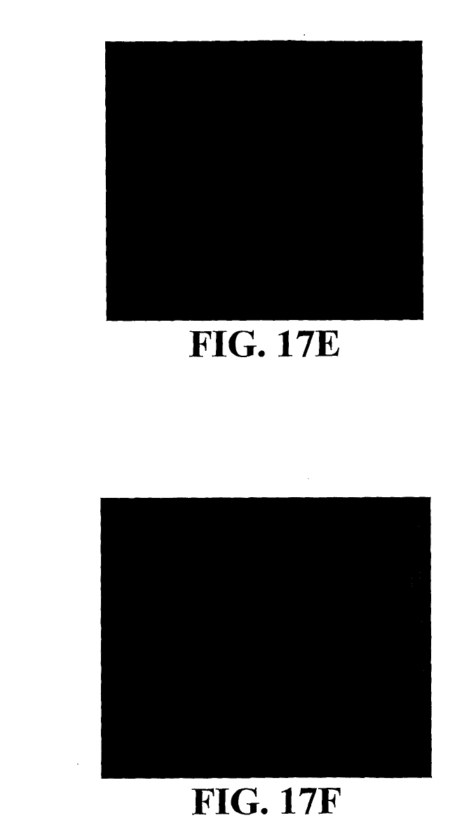

To analyze the biologic activity of human SLLP2, it was determined whether it would bind to an egg, in this case, a mouse egg (FIG. 17, photographs A-F). The results of this experiment demonstrate that human SLLP2 can indeed bind with mouse eggs, i.e., cross-species binding.

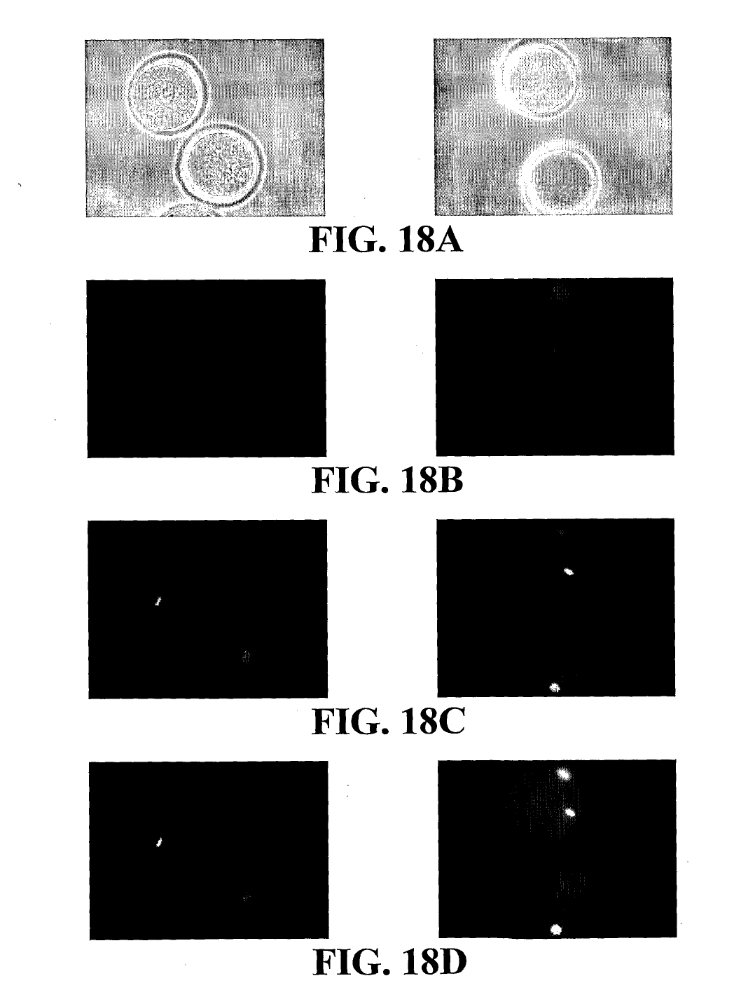

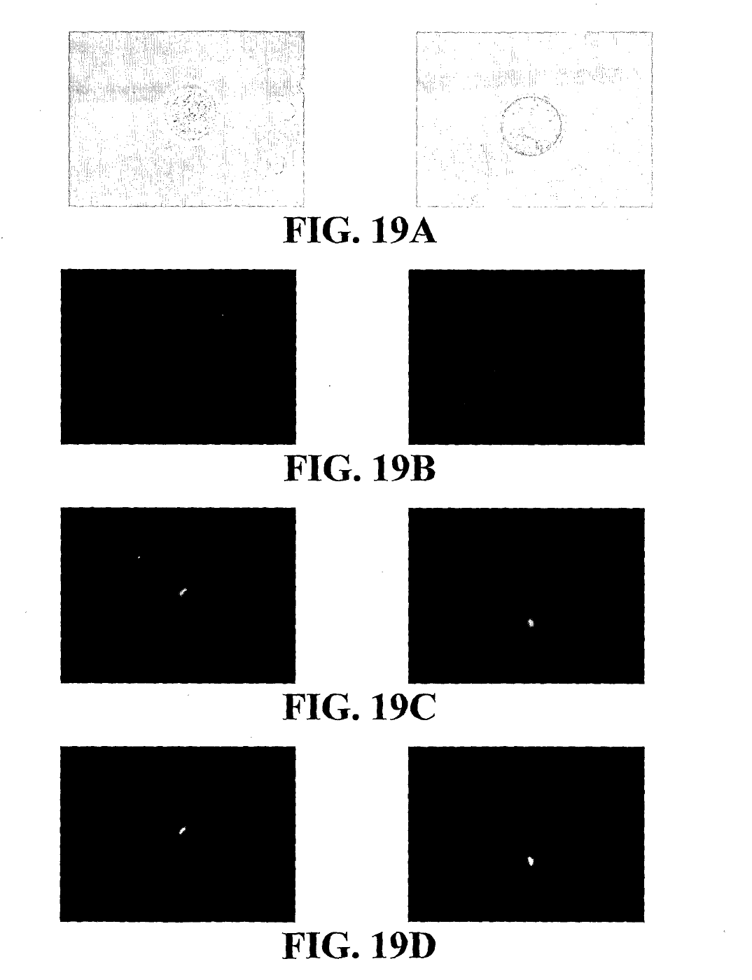

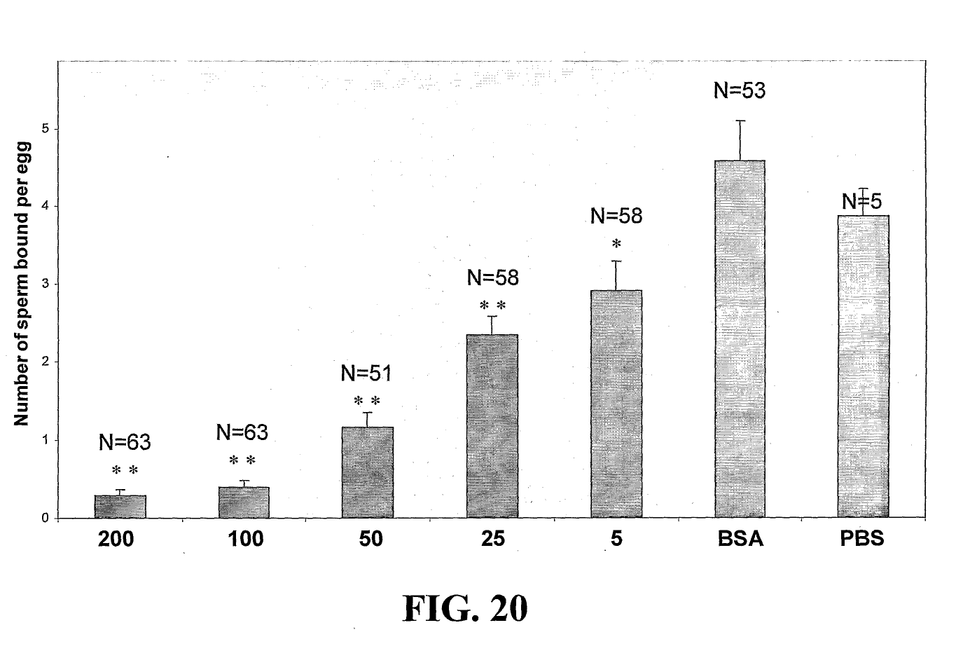

For comparison to the use of human sperm SLLP2 as shown in FIG. 17, an experiment was performed to determine if purified mouse SLLP2 would bind to Zona intact mouse eggs. The results can be seen in the eight photographs of FIG. 18, which demonstrates that the sperm protein mSLLP2 does indeed bind to mouse eggs. The next experiment showed that mouse SLLP2 would bind to Zona-free mouse eggs (see the eight photographs of FIG. 19; left panel (A-D)—no SLLP2; right panel (A-D)—plus SLLP2). Competitive assays were then performed to determine whether the addition of recombinant mouse SLLP2 to a mixture of mouse sperm and mouse eggs could inhibit binding of sperm and eggs. Various amounts of recombinant mouse SLLP2 were added (5, 25, 50, 100, and 200 μg/ml) and the number of sperm bound per egg was determined. It can be seen in FIG. 20 that, relative to BSA (200 μg/ml) or PBS controls, the addition of recombinant mouse SLLP2 was able to reduce the amount of binding occurring between sperm and egg.

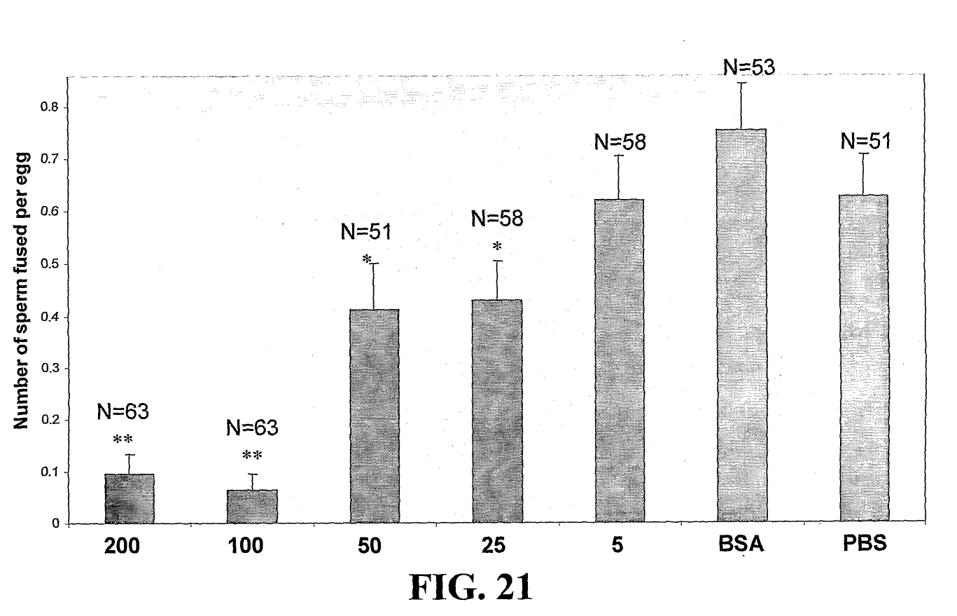

Next, it was determined whether recombinant mouse SLLP2 could inhibit mouse sperm-egg fusion. It can be seen in FIG. 21 that in groups treated with recombinant mouse SLLP2 the number of sperm fused per egg was reduced to about 0.1 sperm fused per egg, at concentrations of 100 or 200 μg/ml recombinant mouse SLLP2, relative to BSA and PBS control treatments.

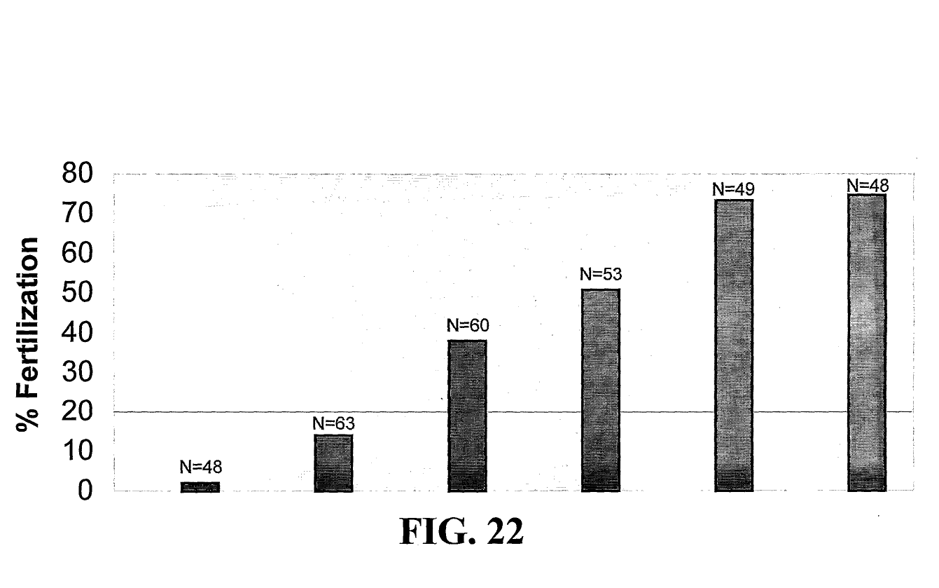

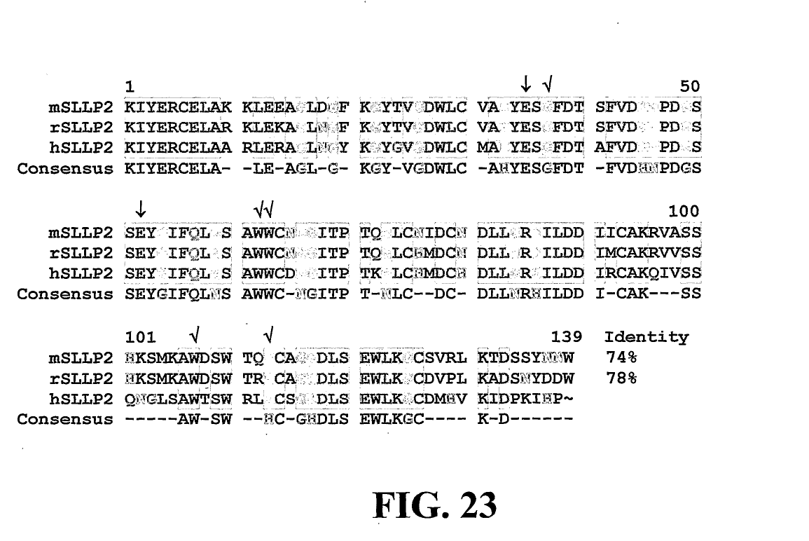

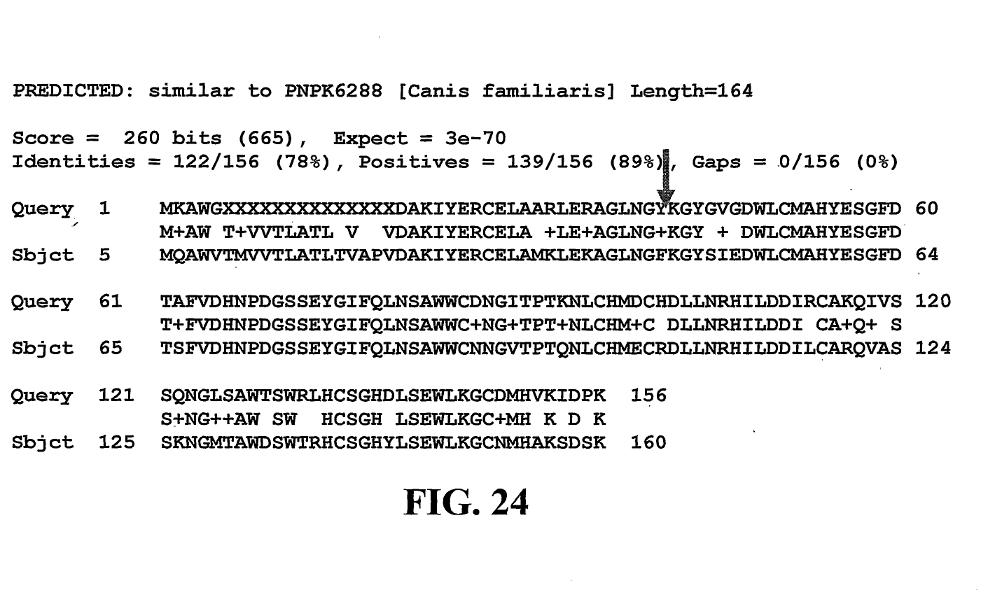

Next, it was determined whether recombinant mouse SLLP2 could actually inhibit or reduce fertilization of mouse eggs with mouse sperm. Recombinant mouse SLLP2 at concentrations of 25, 50, 100, and 200 μg/ml was incubated with sperm and egg, and compared to BSA and PBS controls as described above. It can be seen that fertilization occurred at a rate of about 75% in the BSA and PBS controls groups. However, the addition of rmSLLP2 inhibited fertilization in a dose-dependent fashion, with 200 μg/ml rmSLLP2 nearly totally inhibiting fertilization (FIG. 22). Also provided are comparisons of human SLLP2 to other mammalian sequences (FIG. 23), and well as a comparison indicating the conservation of SLLP2 ortholog in dogs (FIG. 24).

### Example 3

### Identification and Characterization of a Novel Mouse Egg Specific TolA Protein (MET)

This novel egg protein was screened through the protein-protein interaction assay and it was cloned from cDNA library of mouse ovary. This protein appears to be an important pre-patterning protein in mouse eggs and highly regulated in early embryonic development. MET belongs to a TolA (egg specific) family and has a big alanine rich region, which is a feature of this family.

Screening of Putative Egg Receptors for the Mouse Acrosomal Sperm Protein SLLP1 using Biacore

Biacore systems define the characteristics of proteins in terms of their specificity of interaction with other molecules, the rates at which they interact (binding and dissociation), and their affinity (how tightly they bind to another molecule).

The application of Biacore's SPR (Surface plasmon resonance) technology is within the field of proteomics to fish out proteins of interest with subsequent mass spectrometric identification. Purified recombinant SLLP1 was bound on the sensor chip surface and ligand fishing was done with complete mouse egg lysate (1000 zona free eggs) bound to SLLP1, with subsequent identification by mass spectrometry. A number of proteins were screened with the mass spec data and a novel mouse egg specific TolA-like protein (MET) was selected for further characterization, as it was a novel TolA protein and EST data base was very specific to egg and preimplanted embryos. This gene is localized on mouse chromosome number 9 and belongs to the TolA family. The TolA family consists of several bacterial TolA proteins as well as two eukaryotic proteins of currently unknown function. In bacteria, Tol proteins are involved in the translocation of group A colicins. Colicins are bacterial protein toxins, which are active against Escherichia coli and other related species. MET protein is also referred to as a “colicin uptake protein” herein. TolA is anchored to the cytoplasmic membrane by a single membrane spanning segment near the N-terminus, leaving most of the protein exposed to the periplasm.

MET contains a TolA domain and a homologous protein is present in periplasm of E. coli and that how it's presence in mouse eggs seems to be important from evolutionary point of view. Bioinformatic analysis showed that MET has four Protein Kinase C phosphorylation sites, six Casein Kinase II phosphorylation sites and one Tyrosine Kinase phosphorylation site.

Cloning and Expression of MET

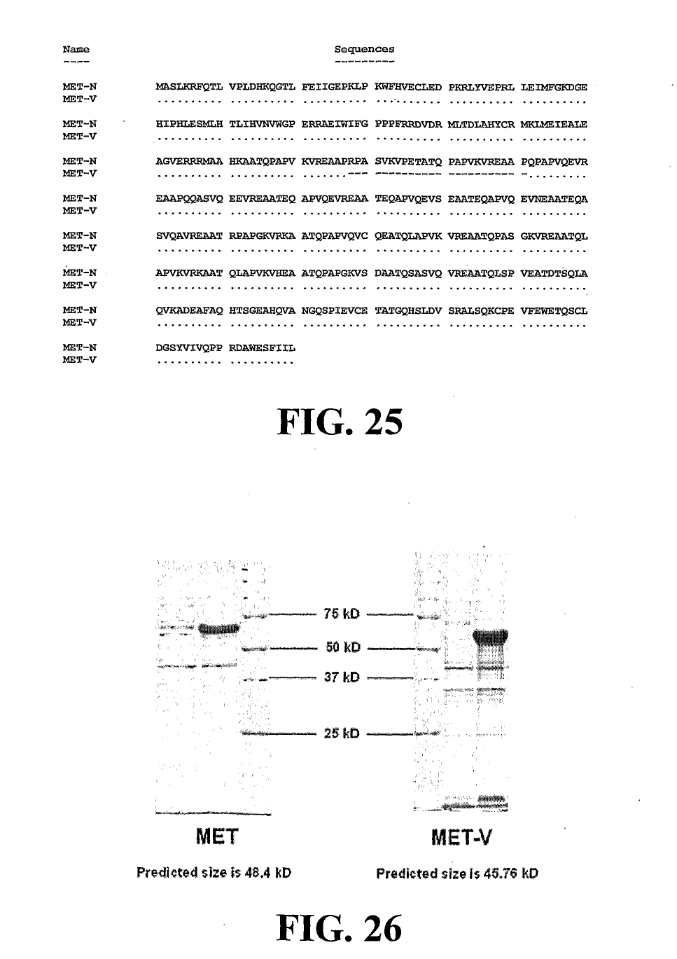

A complete cDNA encoding 440 amino acids was amplified from cDNA library (Ambion) of mouse ovary and cloned in pET expression vector to express this protein as a fusion protein in E. coli (BL21) cells. The mRNA was found to be 1621 bases long. A splice variant of 416 amino acids was found. All the clones were sequenced to check the correct reading frame and a variant was found coding for the same protein with a 24 amino acid fragment deletion. Alignment of the variant with the normal sequence is provided in FIG. 25. The MET nucleic acid (full length/normal and variant) sequences and the protein (full/normal and variant) are as follows:

MET-N Nucleic acid sequence (SEO ID NO:1)
atggcctctctgaagaggtttcagacgctcgtgcccctggatcacaaaca
aggtaccttatttgaaattattggagagcccaagttgcccaagtggttcc
atgtcgaatgcctggaagatccaaaaagactgtacgtggaacctcggcta
ctggaaatcatgtttggtaaggatggagagcacatcccacatcttgaatc
tatgttgcacaccctgatacatgtgaacgtgtggggccctgaaaggcgag
ctgagatttggatattcggaccgccgcctttccgaagggacgttgaccgg
atgctcactgatctggctcactattgccgcatgaaactgatggaaataga
ggctctggaggctggagttgagcgtcgtcgtatggcggcccataaggctg
ccacccagcctgctcccgtgaaggtccgcgaggctgcccctcggcccgct
tccgtgaaggtccctgagacggccacccagcctgctcccgtgaaggtccg
cgaggctgcccctcagcccgctccggtgcaggaggtccgcgaggctgccc
ctcagcaggcttccgtgcaggaggaggtccgcgaggctgccaccgagcag
gctcccgtgcaggaggtccgcgaggctgccaccgagcaggctcccgtgca
ggaggtcagcgaggctgccaccgagcaggctcccgtgcaggaggtcaacg
aggctgccaccgagcaggcttccgtgcaggcggtccgcgaggctgccacc
cggccggctcccgggaaggtccgcaaggcggccacccagccggctccggt
gcaggtttgccaggaggccacccagttggctcccgtgaaggtccgcgagg
cggccacccagccggcttccgggaaggtccgcgaggcggccacccagttg
gctcctgtgaaggtccgcaaggcagccacccagttggctcctgtgaaggt
ccacgaggcggccacccagccggctccggggaaggtcagcgatgctgcca
cgcagtcggcttcggtgcaggttcgtgaggctgccacgcagctgtctccc
gtggaggccactgatactagccagttggctcaggtgaaggctgatgaagc
ctttgcccagcacacttcaggggaggcccaccaggttgccaatgggcagt
ctcccattgaagtctgtgagactgccaccgggcagcattctctagatgtc
tctagggccttgtcccagaagtgtcctgaggtttttgagtgggagaccca
gagttgtttggatggcagctatgtcatagttcagcctccaagggatgcct
gggaatcatttatcatatta
MET-V Nucleic acid sequence (SEQ ID NO:3)
atggcctctctgaagaggtttcagacgctcgtgcccctggatcacaaaca
aggtaccttatttgaaattattggagagcccaagttgcccaagtggttcc
atgtcgaatgcctggaagatccaaaaagactgtacgtggaacctcggcta
ctggaaatcatgtttggtaaggatggagagcacatcccacatcttgaatc
tatgttgcacaccctgatacatgtgaacgtgtggggccctgaaaggcgag
ctgagatttggatattcggaccgccgcctttccgaagggacgttgaccgg
atgctcactgatctggctcactattgccgcatgaaactgatggaaataga
ggctctggaggctggagttgagcgtcgtcgtatggcggcccataaggctg
ccacccagcctgctcccgtgaaggtccgcgaggctgcccctcagcccgct
ccggtgcaggaggtccgcgaggctgcccctcagcaggcttccgtgcagga
ggaggtccgcgaggctgccaccgagcaggctcccgtgcaggaggtccgcg
aggctgccaccgagcaggctcccgtgcaggaggtcagcgaggctgccacc
gagcaggctcccgtgcaggaggtcaacgaggctgccaccgagcaggcttc
cgtgcaggcggtccgcgaggctgccacccggccggctcccgggaaggtcc
gcaaggcggccacccagccggctccggtgcaggtttgccaggaggccacc
cagttggctcccgtgaaggtccgcgaggcggccacccagccggcttccgg
gaaggtccgcgaggcggccacccagttggctcctgtgaaggtccgcaagg
cagccacccagttggctcctgtgaaggtccacgaggcggccacccagccg
gctccggggaaggtcagcgatgctgccacgcagtcggcttcggtgcaggt
tcgtgaggctgccacgcagctgtctcccgtggaggccactgatactagcc
agttggctcaggtgaaggctgatgaagcctttgcccagcacacttcaggg
gaggcccaccaggttgccaatgggcagtctcccattgaagtctgtgagac
tgccaccgggcagcattctctagatgtctctagggccttgtcccagaagt
gtcctgaggtttttgagtgggagacccagagttgtttggatggcagctat
gtcatagttcagcctccaagggatgcctgggaatcatttatcatatta
MET-N Amino Acid Sequence (SEO ID NO:2)
MASLKRFQTLVPLDHKQGTLFEIIGEPKLPKWFHVECLEDPKRLYVEPRL
LEIMFGKDGEHIPHLESMLHTLIHVNVWGPERRAEIWIFGPPPFRRDVDR
MLTDLAHYCRMKLMEIEALEAGVERRRMAAHKAATQPAPVKVREAAPRPA
SVKVPETATQPAPVKVREAAPQPAPVQEVREAAPQQASVQEEVREAATEQ
APVQEVREAATEQAPVQEVSEAATEQAPVQEVNEAATEQASVQAVREAAT
RPAPGKVRKAATQPAPVQVcQEATQLAPVKVREAATQPASGKVREAATQL
APVKVRKAATQLAPVKVHEAATQPAPGKVSDAATQSASVQVREAATQLSP
VEATDTSQLAQVKADEAFAQHTSGEAHQVANGQSPIEVcETATGQHSLDV
SRALSQKCPEVFEWETQSCLDGSYVIVQPPRDAWESFIIL
MET-V Amino Acid sequence (SEO ID NO:4)
MASLKRFQTLVPLDHKQGTLFEIIGEPKLPKWFHVECLEDPKRLYVEPRL
LEIMFGKDGEHIPHLESMLHTLIHVNVWGPERRAEIWIFGPPPFRRDVDR
MLTDLAIIYCRMKLMEIEALEAGVERRRMAAHKAATQPAPVKVREAAPQP
APVQEVREAAPQQASVQEEVRIEAATEQAPVQEVREAATEQAPVQEVSEA
ATEQAPVQEVNEAATEQASVQAVREAATRPAPGKVRKAATQPAPVQVCQE
ATQLAPVKVREAATQPASGKVREAATQLAPVKVRKAATQLAPVKVHEAAT
QPAPGKVSDAATQSASVQVREAATQLSPYEATDTSQLAQVKADEAFAQHT
SGEAHQVANGQSPIEVCETATGQHSLDVSRALSQKCPEVFEWETQSCLDG
SYVIVQPPRDAWESFIIL

Both MET protein sequences were expressed with the pET vector after transformation of bacterial cells with plasmids encoding for fusion proteins, and then induced with 1 mM IPTG (isopropyl-1-thio-β-D-galactopyranoside). The predicted size of the full length protein was 48.4 kD, and the predicted size of the variant with the 24 amino acid deletion was 45.76 kD. Both the expressed and induced proteins were found running comparatively higher on SDS-PAGE than predicted molecular weights (FIG. 26), which could be due to post-translational changes in the bacterial cells.

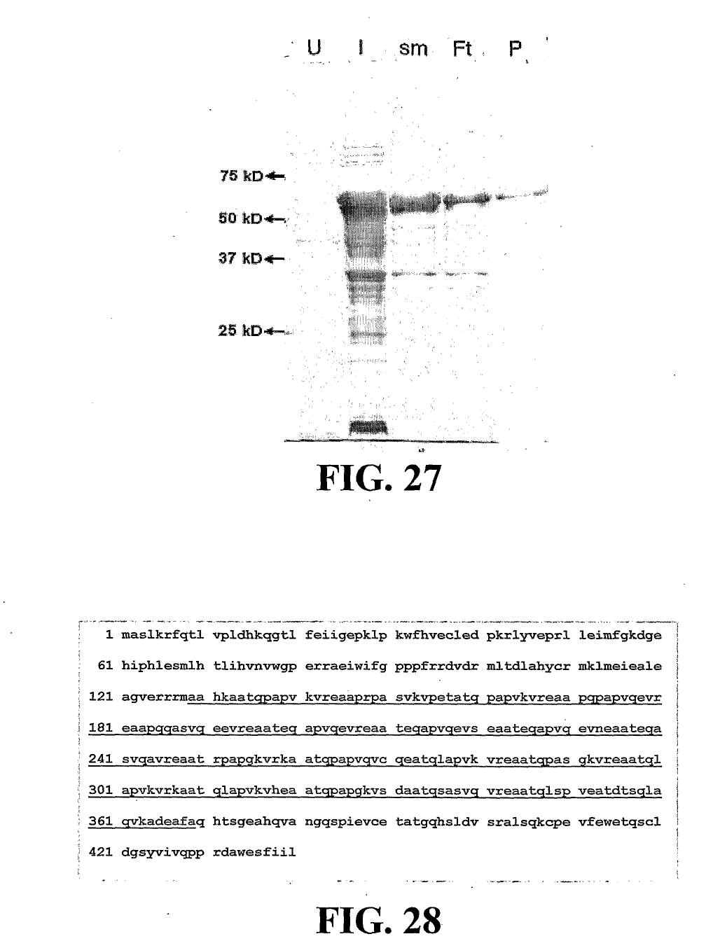

MET was found to accumulate in inclusion bodies. Therefore, bacterial cell lysate was used to purify MET with a Nickel-column, as His-tag of fusion protein bound to Nickel ions. After purification of MET appeared to be a pure single band on SDS-PAGE (FIG. 27). The alanine rich ToLA-like domain in MET has been highlighted in FIG. 28.

Immuno Characterization of Met in Mouse Eggs

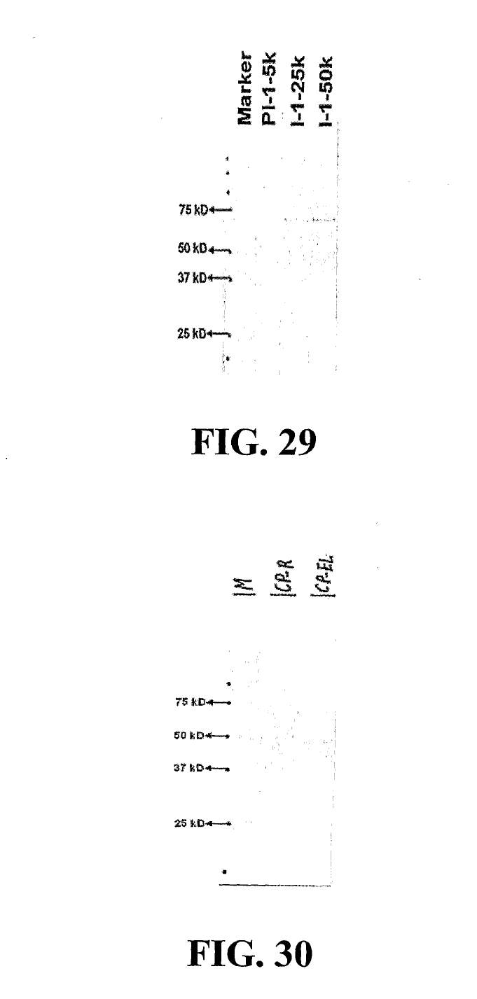

Complete purified MET protein was used to raise antibodies in Guinea pigs. Preimmune screening of these Guinea pigs was done with egg lysate from 100 mouse eggs. Immunization was done in three animals with a primary dose of 150 μg of purified protein, and two more booster doses of 150 μg in three weeks interval. Antibody titer was checked and it was found that 50 ng of purified protein was enough to get good signal, even at 50,000 dilution (FIG. 29). At the same time, two animals were used for adjuvant control—which were negative and did not give any signal with purified recombinant MET.

These antibodies were used to characterize the native form of MET in mouse eggs. Western analysis was done with 100 Zona Intact eggs, using the above antibodies. One strong signal appeared at its predicted size of 48.4 kD (FIG. 30), but at the same time one more faint band appeared as it was in bacterial recombinant of around 65 kD, which proves that the post translational changes are different in bacterial recombinant MET and in it's native form in the mouse eggs.

Localization of Zinc MET in Mouse Eggs

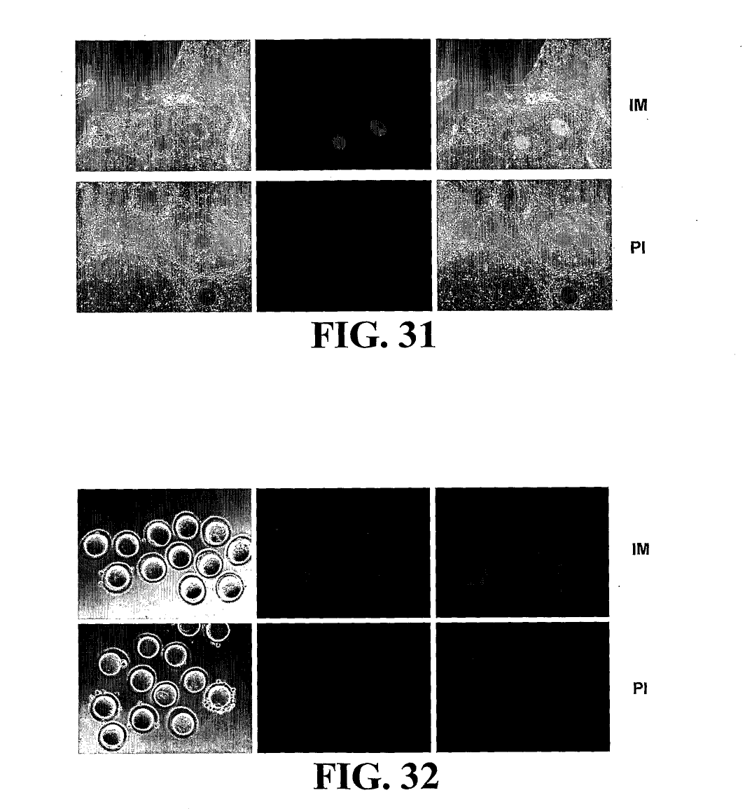

Expression of MET was found to be localized in mouse ovary sections (FIG. 31), and to be very much egg specific protein, without cross-reacting with cumulus cells (FIG. 32). Some permeable fixed eggs were also checked for MET localization and it was observed that MET is abundantly present in the egg cytoplasm. Blastocysts were also checked for MET localization. It was found that MET is expressed in the early blastocyst, but that expression is reduced in the late blastocyst. Such segregation to specific blastomeres may be related to pre-patterning.

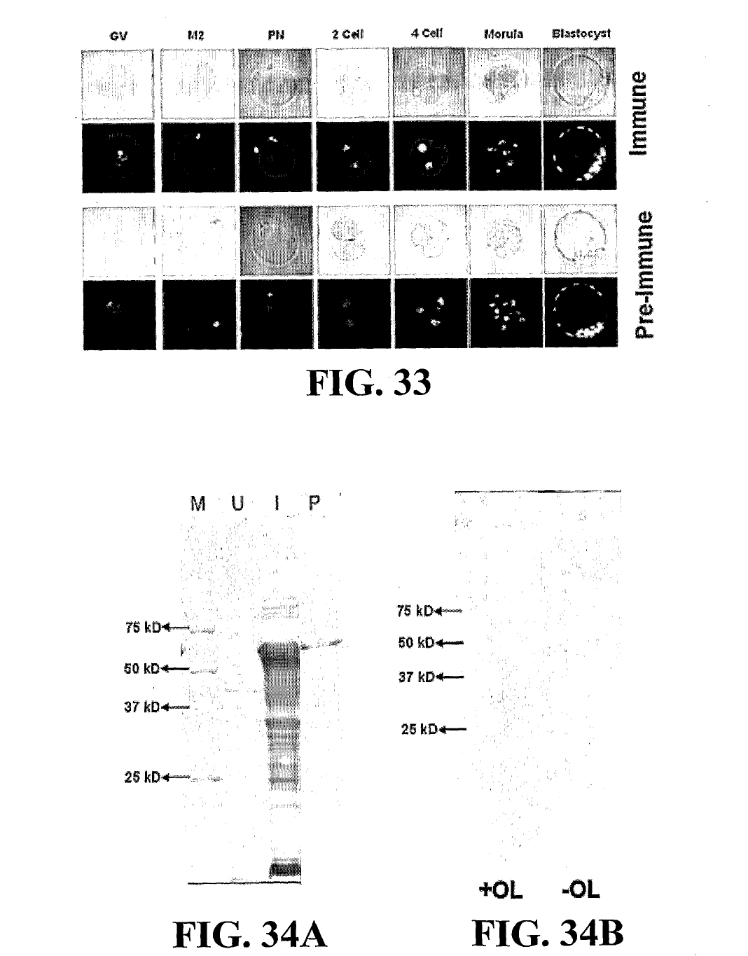

An experiment was designed to examine MET's role during early embryo development and its localization was studied in all the developmental, stages of in-vitro fertilized eggs through the blastocyst stage (FIG. 33). It was found that MET is more abundant at the stage of germinal vesicle and gradually reduced after fertilization, but localization seemed to be very specific at the two cell and four cell stages, and apparently appears to be involved in prepatterning and polarity of embryo. However, in the late blastocyst stage it remained only in the peripheral cells, which can be a marker for trophectoderm cells.

Protein-Protein Interaction of MET and SLLP1 (Far-Western Analysis)

MET was discovered from mouse eggs as an interactive partner of Sperm acrosomal protein SLLP1, therefore a farwestern analysis was designed to prove the evidence of their binding. To that end, 7 μg of recombinant MET was loaded on 12% SDS-PAGE and transferred to nitrocellulose. The membrane was overlaid (OL) with recombinant SLLP1 (2 μg/ml) and probed with anti-SLLP1 monoclonal antibody and secondary antibody (FIGS. 34A and 34B). A strong signal was observed with purified MET, providing further proof that MET binds with SLLP1, and is perhaps an SLLP receptor.

Summary

A full length MET protein and one splice variant were cloned and purified. This protein is a mammalian egg specific TolA protein, and by EST database was shown only in fertilized and unfertilized eggs. According to the known characteristics of the TolA family, MET has a TolA domain of 241 amino acid residues which is highly Alanine rich. It is shown herein that MET is abundant in egg cytoplasm, particularly in cortex. It may segregate to subsets of blastomeres, indicating that MET may provide evidence of pre-patterning and polarity in the egg. MET's localization with specific expression patterns at different developmental stages of egg and embryo proved that it is a very stage specific protein and may be related to pluripotency of the embryonic cells, and later is related to pre-patterning of embryos. Met transcripts and protein are exclusively oocyte and are preimplanted embryo specific, offers a selective window of targeting as an excellent target for contraceptive vaccinogen.

### Example 4

### ZEP, a Novel Egg Protein

The following experiments disclose a novel egg surface receptor (called ZEP below) which interacts with an intra-acrosomal protein, SLLP1. This novel egg receptor for SLLP was screened and identified through a protein-protein interaction assay and was cloned from a mouse ovary cDNA library. This protein appears to be important in sperm binding as well as in early embryonic development (see below). It belongs to a metalloprotease (egg specific) family and has a specific Zinc binding signature. Because it appears to be a Zinc Endopeptidase, it is called ZEP herein.

Further Screening of Putative Egg Receptors which Bind to the Intra Acrosomal Sperm Protein SLLP1 using Biacore

Biacore systems can be used to define the characteristics of proteins in terms of their specificity of interaction with other molecules, the rates at which they interact (binding and dissociation), and their affinity (how tightly they bind to another molecule).

Biacore's SPR (Surface plasmon resonance) technology was used to fish out proteins of interest. Purified recombinant SLLP1 was bound on the sensor chip surface and ligand fishing was done with complete mouse egg lysate (1000 zona free eggs) bound to SLLP1, with subsequent identification by mass spectrometry. Proteins were screened with the mass spectrometry data and ZEP was selected for further characterization as it was a novel metalloprotease and EST data base was very specific to egg and preimplanted embryos.

The gene was localized to mouse chromosome number 2 and belongs to the Astacin family. These proteases require zinc for catalysis and members of this family have an amino terminal propeptide which is cleaved to give the active protease domain.

ZEP showed homology with hatching enzyme EHE7 of Japanese eel Anguilla japonica, therefore it was hypothesized that this protein may be performing a similar function in mouse embryo development. Bioinformatic analysis showed that it has 2 glycosylation sites, phosphorylation sites, and myristylation sites; suggestive of a membrane protein. This protein has a typical zinc-binding region signature and that is how it becomes a zinc-metallopeptidase. Transmembrane topology also predicted a strong transmembrane domain at N-terminal of the protein.

Cloning and Expression of Zinc Endopeptidase (ZEP)

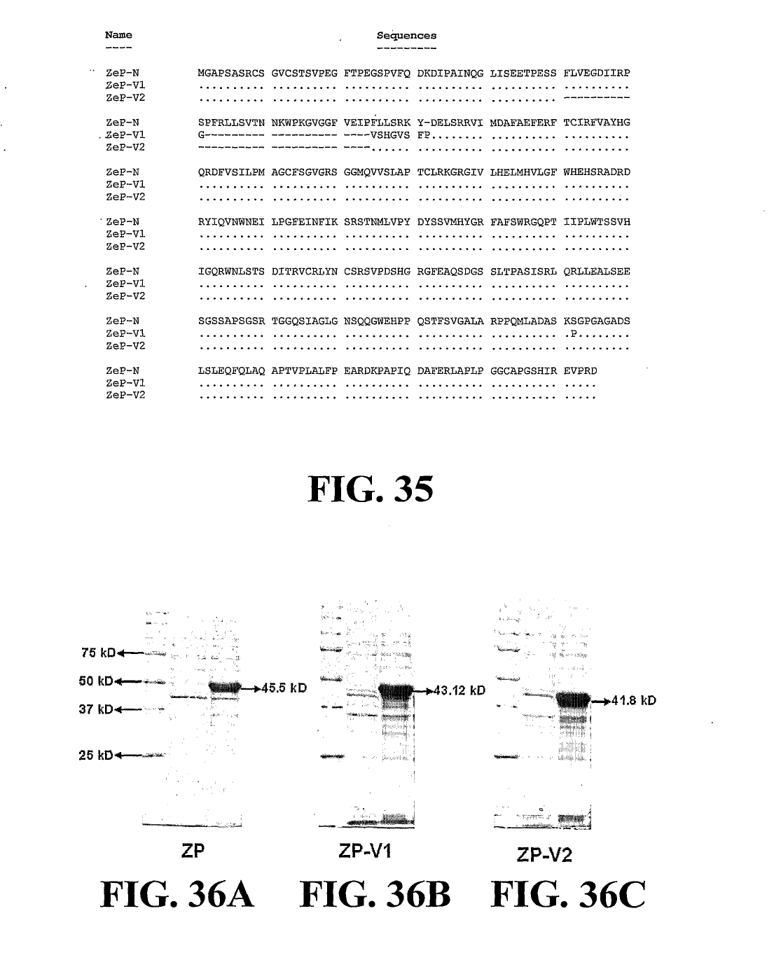

A complete cDNA encoding a 414 amino acid peptide was amplified from cDNA library (Ambion) of mouse ovary and cloned in pET expression vector to express this protein as a fusion protein in E. coli (BL21) cells. All the clones were sequenced to check the correct reading frame and two more variants were found coding for the same protein, one with 34 amino acid deletion and another variant with 34 amino acid deletion and 9 amino acid insertion. Alignment of all the variants (normal and two variants) of Zinc Endopeptidase is given in FIG. 35. The mRNA is 2377 nucleotides long.

All the splice variants were expressed with a pET vector after the transformation of bacterial cells with the plasmids encoding for fusion proteins, and were then induced with 1 mM IPTG (isopropyl-1-thio-β-D-galactopyranoside). Predicted sizes of normal (N) protein was 45.5 kD, variant-1 (V1) with 34 amino acid deletion and 9 amino acid insertion was 43.12 kD and variant-2 (V2) with 34 amino acid deletion was 41.8 kD. All the expressed and induced proteins were in an SDS-PAGE analysis with their expected size (FIG. 36A-C; left gel—ZEP; middle gel—ZP V-1; right gel—ZP V-2).

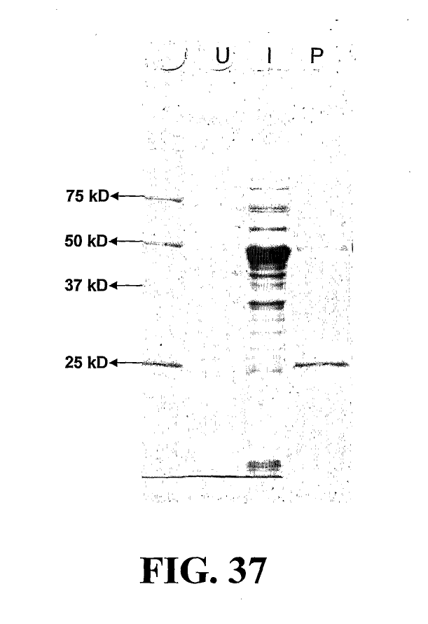

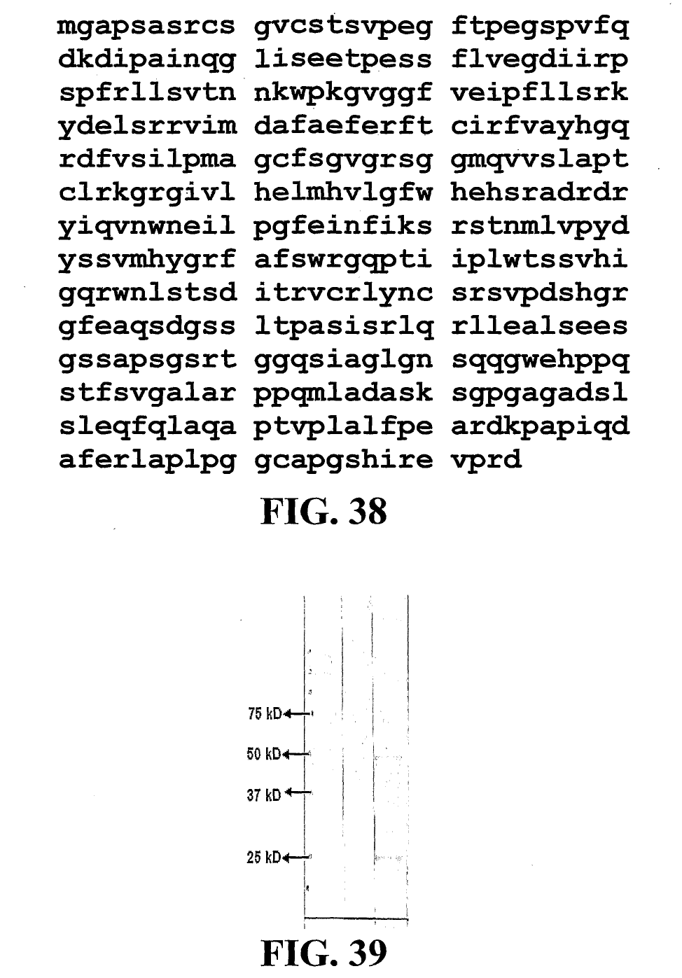

This protein was found to accumulate in inclusion bodies. Therefore, bacterial cell lysate was used to purify this protein with Nickel-column, as His-tag of fusion protein bound to Nickel ions. After purification of ZEP protein, it was observed that there was an autolytic cleavage and two bands were detected by SDS-PAGE, including a new lower molecular weight band of about 25 kD (FIG. 37; see all four lanes of the gel). N-terminal sequencing was done on this lower band and the cleavage site was found after 204 amino acids (see sequence of FIG. 38). It is not yet clear how this protein is cleaved at this particular site, but the sequence data of FIG. 38 explain the transmembrane domain, zinc-binding signature and the cleavage site in the amino acid sequence of ZEP. Additionally, an analysis of the ZEP amino acid sequence further suggested a transmembrane structure (as indicated graphically—not shown: TMpred output suggested a transmembrane topology with the preferred model comprising the N-terminus outside with one strong transmembrane helices, and a total score: 755 o-i 122-152 (31)).

The nucleic acid and amino acid sequences of the normal (N) ZEP and the two variants (V1 and V2) disclosed herein are as follows:

ZEP-N Nucleic acid sequence (SEQ ID NO:5)
atgggagcaccctcagcatccagatgttctggagtctgcagtaccagtgt
tccagaaggcttcactcctgagggaagcccggtatttcaggacaaggaca
tccccgcaattaaccaagggctcatctcagaggagaccccagaaagcagc
ttcctggtagaaggggacattatccggccaagccctttccgattgttgtc
agtgaccaataataaatggcccaagggcgttggtggctttgtggagatcc
ccttcctgctttccagaaagtatgatgaactcagccgccgggtcattatg
gatgcctttgctgagtttgaacgtttcacatgcatccggtttgttgccta
ccatggtcagagagactttgtttccattcttcctatggcggggtgtttct
ctggtgtgggacgcagtggagggatgcaggtggtgtccttggcacccact
tgtctccggaagggccgaggcattgtcctacatgagctcatgcacgtact
tggcttctggcatgagcattcacgggcagatcgggaccgctacatccaag
tcaactggaacgagatcctcccgggctttgaaatcaacttcatcaagtca
cggagtaccaatatgttagttccctatgactactcatctgtgatgcatta
tgggagatttgccttcagctggcgtgggcagcccaccatcataccactct
ggacctccagtgttcacattggccagcgatggaacctgagtacctcagat
atcacccgggtctgcaggctgtataactgcagccggagtgtccctgactc
ccacgggagagggtttgaggcccagagtgatggaagcagcctcacccctg
cctctatatcacgtctacaaagacttctcgaggcactgtcagaggaatct
ggaagctctgcccctagtggctccaggactggaggccagagtattgccgg
gcttggtaacagccagcaaggatgggagcatcctcctcagagcacattca
gtgtgggagccttggcaagaccacctcagatgctagccgatgcttcaaaa
tcggggcctggagcaggtgcagacagcttgtctctagagcagttccagct
agcccaggcccccactgtacctcttgctctatttccagaagccagagaca
agccagcacctatccaagatgcctttgagaggctagctccacttccagga
ggctgtgcacctggaagtcacattagagaggtgcccagagac
ZEP-V1 Nucleic acid sequence (SEQ ID NO:7)
atgggagcaccctcagcatccagatgttctggagtctgcagtaccagtgt
tccagaaggcttcactcctgagggaagcccggtatttcaggacaaggaca
tccccgcaattaaccaagggctcatctcagaggagaccccagaaagcagc
ttcctggtagaaggggacattatccggccaggggtcagccacggtgtgtc
tttcccagatgaactcagccgccgggtcattatggatgcctttgctgagt
ttgaacgtttcacatgcatccggtttgttgcctaccatggtcagagagac
tttgtttccattcttcctatggcggggtgtttctctggtgtgggacgcag
tggagggatgcaggtggtgtccttggcacccacttgtctccggaagggcc
gaggcattgtcctacatgagctcatgcacgtacttggcttctggcatgag
cattcacgggcagatcgggaccgctacatccaagtcaactggaacgagat
cctcccgggctttgaaatcaacttcatcaagtcacggagtaccaatatgt
tagttccctatgactactcatctgtgatgcattatgggagatttgccttc
agctggcgtgggcagcccaccatcataccactctggacctccagtgttca
cattggccagcgatggaacctgagtacctcagatatcacccgggtctgca
ggctgtataactgcagccggagtgtccctgactcccacgggagagggttt
gaggcccagagtgatggaagcagcctcacccctgcctctatatcacgtct
acaaagacttctcgaggcactgtcagaggaatctggaagctctgccccta
gtggctccaggactggaggccagagtattgccgggcttggtaacagccag
caaggatgggagcatcctcctcagagcacattcagtgtgggagccttggc
aagaccacctcagatgctagccgatgcttcaaaatcggggcctggagcag
gtgcagacagcttgtctctagagcagttccagctagcccaggcccccact
gtacctcttgctctatttccagaagccagagacaagccagcacctatcca
agatgcctttgagaggctagctccacttccaggaggctgtgcacctggaa
gtcacattagagaggtgcccagagac
ZEP-V2 Nucleic acid sequence (SEQ ID NO:9)
atgggagcaccctcagcatccagatgttctggagtctgcagtaccagtgt
tccagaaggcttcactcctgagggaagcccggtatttcaggacaaggaca
tccccgcaattaaccaagggctcatctcagaggagaccccagaaagcagc
ttcctgctttccagaaagtatgatgaactcagccgccgggtcattatgga
tgcctttgctgagtttgaacgtttcacatgcatccggtttgttgcctacc
atggtcagagagactttgtttccattcttcctatggcggggtgtttctct
ggtgtgggacgcagtggagggatgcaggtggtgtccttggcacccacttg
tctccggaagggccgaggcattgtcctacatgagctcatgcacgtacttg
gcttctggcatgagcattcacgggcagatcgggaccgctacatccaagtc
aactggaacgagatcctcccgggctttgaaatcaacttcatcaagtcacg
gagtaccaatatgttagttccctatgactactcatctgtgatgcattatg
ggagatttgccttcagctggcgtgggcagcccaccatcataccactctgg
acctccagtgttcacattggccagcgatggaacctgagtacctcagatat
cacccgggtctgcaggctgtataactgcagccggagtgtccctgactccc
acgggagagggtttgaggcccagagtgatggaagcagcctcacccctgcc
tctatatcacgtctacaaagacttctcgaggcactgtcagaggaatctgg
aagctctgcccctagtggctccaggactggaggccagagtattgccgggc
ttggtaacagccagcaaggatgggagcatcctcctcagagcacattcagt
gtgggagccttggcaagaccacctcagatgctagccgatgcttcaaaatc
ggggcctggagcaggtgcagacagcttgtctctagagcagttccagctag
cccaggcccccactgtacctcttgctctatttccagaagccagagacaag
ccagcacctatccaagatgcctttgagaggctagctccacttccaggagg
ctgtgcacctggaagtcacattagagaggtgcccagagac
ZEP-N Amino acid sequence (SEQ ID NO:6)
MGAPSASRCSGVCSTSVPEGFTPEGSPVFQDKDIPAINQGLISEETPESS
FLVEGDIIRPSPFRLLSVTNNKWPKGVGGFVEIPFLLSRKYDELSRRVIM
DAFAEFERFTCIRFVAYHGQRDFVSILPMAGCFSGVGRSGGMQVVSLAPT
CLRKGRGIVLHELMHVLGFWHEHSRADRDRYIQVNWNEILPGFEINFIKS
RSTNMLVPYDYSSVMHYGRFAFSWRGQPTIIPLWTSSVHIGQRWNLSTSD
ITRVCRLYNCSRSVPDSHGRGFEAQSDGSSLTPASISRLQRLLEALSEES
GSSAPSGSRTGGQSIAGLGNSQQGWEHPPQSTFSVGALARPPQMLADASK
SGPGAGADSLSLEQFQLAQAPTVPLALFPEARDKPAPIQDAFERLAPLPG
GCAPGSHIREVPRD
ZEP-V1 Amino acid sequence (SEQ ID NO:8)
MGAPSASRCSGVCSTSVPEGFTPEGSPVFQDKDIPAINQGLISEETPESS
FLVEGDIIRPGVSHGVSFPDELSRRVIMDAFAFEERFTCIRFVAYHGQRD
FVSILPMAGCFSGVGRSGGMQVVSLAPTCLRKGRGIVLHELMHVLGFWII
EHSRADRDRYIQVNWNEILPGFEINFIKSRSTNMLVPYDYSSVMHYGRFA
FSWRGQPTIIPLWTSSVHIGQRWNLSTSDITRVCRLYNCSRSVPDSHGRG
FEAQSDGSSLTPASISRLQRLLEALSEESGSSAPSGSRTGGQSIAGLGNS
QQGWEHPPQSTFSVGALARPPQMLADASKPGPGAGADSLSLEQFQLAQAP
TVPLALFPEARDKPAPIQDAFERLAPLPGGCAPGSHIREVPRD
ZEP-V2 Amino acid sequence (SEQ ID NO:10)
MGAPSASRCSGVCSTSVPEGFTPEGSPVFQDKDIPAINQGLISEETPESS
FLLSRKYDELSRRVIMDAFAEFERFTCIRFVAYHGQRDFVSILPMAGCFS
GVGRSGGMQVVSLAPTCLRKGRGIVLHELMHVLGFWHEHSRADRDRYIQV
NWNEILPGFEINFIKSRSTNMLVPYDYSSVMHYGRFAFSWRGQPTIIPLW
TSSVHIGQRWNLSTSDITRVCRLYNCSRSVPDSHGRGFEAQSDGSSLTPA
SISRLQRLLEALSEESGSSAPSGSRTGGQSIAGLGNSQQGWEHPPQSTFS
VGALARPPQMLADASKSGPGAGADSLSLEQFQLAQAPTVPLALFPEARDK
PAPIQDAFERLAPLPGGCAPGSHIREVPRD

Immuno Characterization of Zinc Endopeptidase (ZEP) in Mouse Eggs

Purified protein was used to raise antibodies in Guinea pigs. Preimmune screening of these Guinea pigs was done with egg lysate of 100 mouse eggs. Immunization was done in three animals with primary dose of 150 μg of purified protein and two more booster doses of 150 μg in three weeks interval. Antibody titer was checked and it was found that 50 ng of purified protein was enough to get good signal even at 50,000 dilution (see Western blot of Recombinant ZEP, FIG. 39). Concurrently, two animals were used for adjuvant control, which proved to be negative and did not give any signal with purified recombinant ZEP.

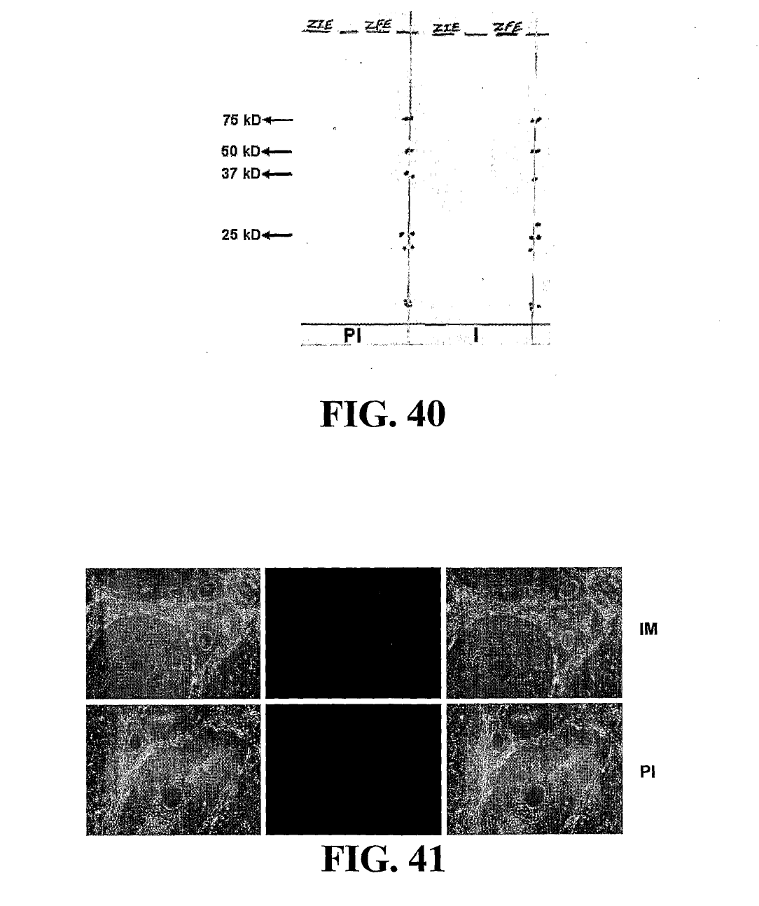

The antibodies obtained as described were used to characterize the native form of this protein in mouse eggs. Western analysis was done with 150 zona intact and 150 zona free eggs, using the above antibodies as an immune sera screening assay. A signal was found at about 45.5 kD (FIG. 40), in both the zona intact and zona free eggs. However, two more bands, which migrated at about 50 kD and 32 kD, were also observed. It is possible that this protein exists in different forms in the eggs, or different spliced variants may be coding for different sizes of similar proteins.

Localization of Zinc Endopeptidase (ZEP) in Mouse Eggs

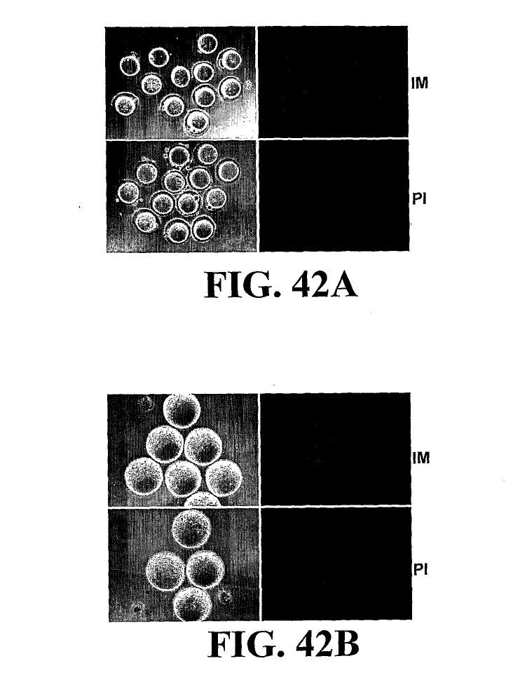

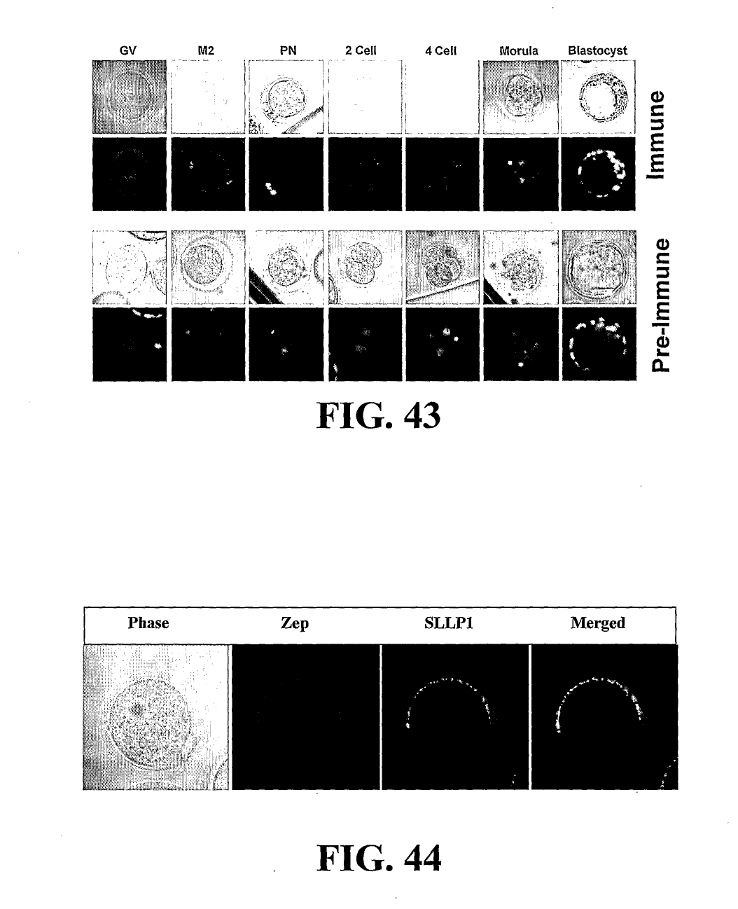

It was found that ZEP is egg specific and does not cross react with cumulus cells (FIGS. 41 and 42). In FIG. 41 it can be seen that immunolocalization of zinc-peptidase in ovary sections is localized, and is very much egg specific, including secondary and tertiary follicles. It was further observed that ZEP is localized on the egg surface in the microvillar region. Some blastocysts were also checked for the ZEP localization. In the eight panels of FIG. 42 (4 each in 42a and 42b), it can be seen in the photographs that ZEP is located on the egg surface in the microvillar region. Also found was a faint signal in the form of patches. To determine ZEP's developmental regulation, its localization was checked in all the developmental stages of in-vitro fertilized eggs through the blastocyst stage (FIG. 43) and it was found that ZEP is more abundant at the stage of germinal vesicle and gradually reduced after fertilization. In the blastocyst stage it remained in only some of the peripheral cells.

Protein-Protein Interaction of ZEP and SLLP1

ZEP was picked up from mouse eggs as an interactive partner of the sperm acrosomal protein SLLP1. Therefore, to verify the interaction of ZEP and SLLP1 in the native form of the eggs; co-localization was assayed on the egg surface. Mouse eggs were incubated with 10 μg/ml of recombinant SLLP1 for an hour and then with ZEP and SLLP1 antibodies simultaneously for another hour. A secondary antibody of ZEP was cy3 conjugated and a secondary antibody of SLLP1 was FITC conjugated. To compare localization, images were captured separately and merged after that (see the four confocal images of co-localization of ZEP and SLLP1 on mouse eggs in FIG. 44). The ZEP signal was present only in the microvillar region, whereas the major signal for SLLP1 was located in microvillar region and spread little bit in the perivitelline space. The data demonstrate that the two proteins are binding with one another, because their signal is completely merged (see panel four of FIG. 44).

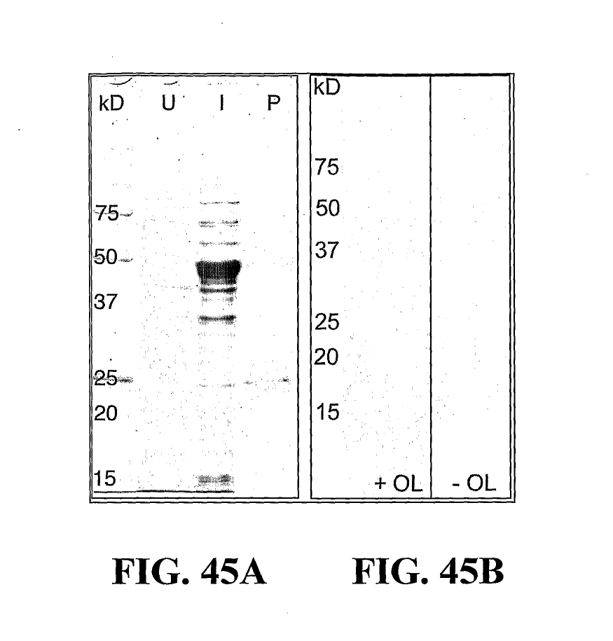

To further confirm binding, farwestern analyses were performed to further demonstrate that SLLP1 binds with ZEP. To that end, 7.0 μg of recombinant ZEP was loaded onto a 12% SDS gel, subjected to PAGE, and transferred to a nitrocellulose membrane (see FIG. 45, left panel). The membrane was overlaid (OL) with recombinant SLLP1 (2.0 μg/ml) and probed with anti-SLLP1 monoclonal antibody and secondary antibody (FIG. 45; right panel). A signal was observed with the upper band of ZEP, but not with the lower band. These data suggest that the N-terminal of ZEP has the SLLP1 binding capability, and further suggest why it does not bind with the C-terminal band when the N-terminal is truncated.

### SUMMARY

The present invention discloses a full length ZEP and two splice variants. This protein is a mammalian egg specific Zinc Peptidase. In summary, ZEP is localized at the egg membrane at different developmental stage. Co-localization with SLLP1 at the egg membrane further demonstrates that it binds with Sperm Acrosomal protein SLLP1. Because of the sperm binding capability of ZEP, and its stage specific expression, this protein seems to be important for fertilization and an excellent target for contraceptive vaccinogen.

Other methods which were used but not described herein are well known and within the competence of one of ordinary skill in the art of cell biology, molecular biology, and medicine.

The invention should not be construed to be limited solely to the assays and methods described herein, but should be construed to include other methods and assays as well. One of skill in the art will know that other assays and methods are available to perform the procedures described herein.

Headings are included herein for reference and to aid in locating certain sections. These headings are not intended to limit the scope of the concepts described therein under, and these concepts may have applicability in other sections throughout the entire specification.

The disclosures of each and every patent, patent application, and publication cited herein are hereby incorporated herein by reference in their entirety.

While this invention has been disclosed with reference to specific embodiments, it is apparent that other embodiments and variations of this invention may be devised by the previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. The appended claims are intended to be construed to include all such embodiments and equivalent variations. Accordingly, the present invention is not intended to be limited to the embodiments shown herein but is to be accorded the widest scope consistent with the principles and novel features disclosed herein.

What is claimed is:

1. A composition for preventing or inhibiting a sperm from binding to an egg, said composition comprising a pharmaceutically-acceptable carrier and at least one isolated nucleic acid comprising a nucleic acid sequence encoding a protein, or a variant, homolog, or fragment thereof, wherein said protein comprises an amino acid sequence selected from the group consisting of SEQ ID NOS:2, 4, 6, 8, 10, 12, 14, 16, and 18.

2. The composition of claim 1, wherein said nucleic acid sequence is selected from the group consisting of SEQ ID NOS:1, 3, 5, 7, 9, 11, 13, 15, and 17.

3. A composition for preventing or inhibiting a sperm from fusing with an egg, said composition comprising a pharmaceutically-acceptable carrier and at least one isolated nucleic acid comprising a nucleic acid sequence encoding a protein, or a variant, homolog, or fragment thereof, wherein said protein comprises an amino acid sequence selected from the group consisting of SEQ ID NOS:2, 4, 6, 8, 10, 12, 14, 16, and 18.

4. The composition of claim 3, wherein said nucleic acid sequence is selected from the group consisting of SEQ ID NOS:1, 3, 5, 7, 9, 11, 13, 15, and 17.

5. A composition for preventing or inhibiting fertilization, said composition comprising a pharmaceutically-acceptable carrier and at least one isolated nucleic acid comprising a nucleic acid sequence encoding a protein, or a variant, homolog, or fragment thereof, wherein said protein comprises an amino acid sequence selected from the group consisting of SEQ ID NOS:2, 4, 6, 8, 10, 12, 14, 16, and 18.

6. The composition of claim 5, wherein said nucleic acid sequence is selected from the group consisting of SEQ ID NOS:1, 3, 5, 7, 9, 11, 13, 15, and 17.

7. The composition of claim 5, wherein said fertilization is inhibited in vivo.

8. The composition of claim 7, wherein said in vivo inhibition of fertilization is in a human.

9. A method of preventing or inhibiting fertilization, said method comprising contacting a sperm or egg with an effective amount of at least one compound which prevents or inhibits the interaction of a sperm protein with an egg protein.

10. The method of claim 9, wherein said compound prevents or inhibits the interaction of a sperm protein with a MET protein, further wherein said MET protein comprises an amino acid sequence selected from the group consisting of SEQ ID NOs:2 and 4, or variants, homologs, or fragments thereof.

11. The method of claim 9, wherein said compound prevents or inhibits the interaction of a sperm protein with a ZEP protein, further wherein said ZEP protein comprises an amino acid sequence selected from the group consisting of SEQ ID NOs:6, 8, and 10, or variants, homologs, or fragments thereof.

12. The method of claim 9, wherein said compound is selected from the group consisting of an antibody directed against an SLLP1 protein, an antibody directed against an SLLP2 protein, an antibody directed against a MET protein, an antibody directed against a ZEP protein, a recombinant SLLP1 protein, or a biologically active variant, homolog, or fragment thereof, a recombinant SLLP2 protein, or a biologically active variant, homolog, or fragment thereof, a recombinant MET protein, or a biologically active variant, homolog, or fragment thereof, and a recombinant ZEP protein or a biologically active variant, homolog, or fragment thereof.

13. The method of claim 12, wherein said protein comprises an amino acid sequence selected from the group consisting of SEQ ID NOs:2, 4, 6, 8, 10, 12, 14, 16, and 18.

14. The method of claim 12, wherein said antibody directed against an SLLP1 protein is a monoclonal antibody.

15. The method of claim 12, wherein said antibody directed against an SLLP2 protein is a monoclonal antibody.

16. The method of claim 12, wherein said antibody directed against a MET protein is a monoclonal antibody.

17. The method of claim 12, wherein said antibody directed against a ZEP protein is a monoclonal antibody.

18. The method of claim 9, wherein interaction is binding between a sperm protein and an egg protein.

19. A pharmaceutical composition comprising a pharmaceutically-acceptable carrier and at least one egg protein, or a variant, homolog, fragment or derivative thereof, wherein said protein is capable of inducing an immune response useful for preventing or inhibiting conception in a subject, further wherein said at least one egg protein comprises an amino acid sequence selected from the group consisting of SEQ ID NOs:2, 4, 6, 8, and 10.

20. The pharmaceutical composition of claim 19, wherein said composition comprises at least two egg proteins.

21. The pharmaceutical composition of claim 19, wherein said composition further comprises an additional egg protein.

22. The pharmaceutical composition of claim 19, wherein said protein capable of eliciting an immune response useful for preventing or inhibiting conception, prevents or inhibits fertilization.

23. A kit for preventing or inhibiting conception in a subject, said kit comprising the pharmaceutical composition of claim 19, an applicator, and an instructional material for the use thereof.

24. A kit for preventing or inhibiting fertilization, said kit comprising the pharmaceutical composition of claim 5, an applicator, and an instructional material for the use thereof.

25. A method for preventing or inhibiting fertilization, said method comprising contacting an egg with an effective amount of an inhibitor of MET protein.

26. The method of claim 25, wherein said inhibitor of MET protein inhibits MET protein synthesis, expression, or function.

27. The method of claim 25, wherein said MET protein comprises an amino acid sequence selected from the group consisting of SEQ ID NOs:2 and 4.

28. A method for preventing or inhibiting fertilization, said method comprising contacting an egg with an effective amount of an inhibitor of ZEP protein.

29. The method of claim 28, wherein said inhibitor of ZEP protein inhibits ZEP protein synthesis, expression, or function.

30. The method of claim 28, wherein said ZEP protein comprises an amino acid sequence selected from the group consisting of SEQ ID NOs:6, 8, and 10.


SEQUENCE LISTING (18 sequences)

SEQ ID NO: 1 - DNA, 1320 bp, mus musculus
atggcctctctgaagaggtttcagacgctcgtgcccctggatcacaaacaaggtaccttatttgaaattattggagagcccaagttgcccaagtggttccatgtcgaatgcctggaagatccaaaaagactgtacgtggaacctcggctactggaaatcatgtttggtaaggatggagagcacatcccacatcttgaatctatgttgcacaccctgatacatgtgaacgtgtggggccctgaaaggcgagctgagatttggatattcggaccgccgcctttccgaagggacgttgaccggatgctcactgatctggctcactattgccgcatgaaactgatggaaatagaggctctggaggctggagttgagcgtcgtcgtatggcggcccataaggctgccacccagcctgctcccgtgaaggtccgcgaggctgcccctcggcccgcttccgtgaaggtccctgagacggccacccagcctgctcccgtgaaggtccgcgaggctgcccctcagcccgctccggtgcaggaggtccgcgaggctgcccctcagcaggcttccgtgcaggaggaggtccgcgaggctgccaccgagcaggctcccgtgcaggaggtccgcgaggctgccaccgagcaggctcccgtgcaggaggtcagcgaggctgccaccgagcaggctcccgtgcaggaggtcaacgaggctgccaccgagcaggcttccgtgcaggcggtccgcgaggctgccacccggccggctcccgggaaggtccgcaaggcggccacccagccggctccggtgcaggtttgccaggaggccacccagttggctcccgtgaaggtccgcgaggcggccacccagccggcttccgggaaggtccgcgaggcggccacccagttggctcctgtgaaggtccgcaaggcagccacccagttggctcctgtgaaggtccacgaggcggccacccagccggctccggggaaggtcagcgatgctgccacgcagtcggcttcggtgcaggttcgtgaggctgccacgcagctgtctcccgtggaggccactgatactagccagttggctcaggtgaaggctgatgaagcctttgcccagcacacttcaggggaggcccaccaggttgccaatgggcagtctcccattgaagtctgtgagactgccaccgggcagcattctctagatgtctctagggccttgtcccagaagtgtcctgaggtttttgagtgggagacccagagttgtttggatggcagctatgtcatagttcagcctccaagggatgcctgggaatcatttatcatatta

SEQ ID NO: 2 - protein, 440 aa, mus musculus
MASLKRFQTLVPLDHKQGTLFEIIGEPKLPKWFHVECLEDPKRLYVEPRLLEIMFGKDGEHIPHLESMLHTLIHVNVWGPERRAEIWIFGPPPFRRDVDRMLTDLAHYCRMKLMEIEALEAGVERRRMAAHKAATQPAPVKVREAAPRPASVKVPETATQPAPVKVREAAPQPAPVQEVREAAPQQASVQEEVREAATEQAPVQEVREAATEQAPVQEVSEAATEQAPVQEVNEAATEQASVQAVREAATRPAPGKVRKAATQPAPVQVCQEATQLAPVKVREAATQPASGKVREAATQLAPVKVRKAATQLAPVKVHEAATQPAPGKVSDAATQSASVQVREAATQLSPVEATDTSQLAQVKADEAFAQHTSGEAHQVANGQSPIEVCETATGQHSLDVSRALSQKCPEVFEWETQSCLDGSYVIVQPPRDAWESFIIL

SEQ ID NO: 3 - DNA, 1248 bp, mus musculus
atggcctctctgaagaggtttcagacgctcgtgcccctggatcacaaacaaggtaccttatttgaaattattggagagcccaagttgcccaagtggttccatgtcgaatgcctggaagatccaaaaagactgtacgtggaacctcggctactggaaatcatgtttggtaaggatggagagcacatcccacatcttgaatctatgttgcacaccctgatacatgtgaacgtgtggggccctgaaaggcgagctgagatttggatattcggaccgccgcctttccgaagggacgttgaccggatgctcactgatctggctcactattgccgcatgaaactgatggaaatagaggctctggaggctggagttgagcgtcgtcgtatggcggcccataaggctgccacccagcctgctcccgtgaaggtccgcgaggctgcccctcagcccgctccggtgcaggaggtccgcgaggctgcccctcagcaggcttccgtgcaggaggaggtccgcgaggctgccaccgagcaggctcccgtgcaggaggtccgcgaggctgccaccgagcaggctcccgtgcaggaggtcagcgaggctgccaccgagcaggctcccgtgcaggaggtcaacgaggctgccaccgagcaggcttccgtgcaggcggtccgcgaggctgccacccggccggctcccgggaaggtccgcaaggcggccacccagccggctccggtgcaggtttgccaggaggccacccagttggctcccgtgaaggtccgcgaggcggccacccagccggcttccgggaaggtccgcgaggcggccacccagttggctcctgtgaaggtccgcaaggcagccacccagttggctcctgtgaaggtccacgaggcggccacccagccggctccggggaaggtcagcgatgctgccacgcagtcggcttcggtgcaggttcgtgaggctgccacgcagctgtctcccgtggaggccactgatactagccagttggctcaggtgaaggctgatgaagcctttgcccagcacacttcaggggaggcccaccaggttgccaatgggcagtctcccattgaagtctgtgagactgccaccgggcagcattctctagatgtctctagggccttgtcccagaagtgtcctgaggtttttgagtgggagacccagagttgtttggatggcagctatgtcatagttcagcctccaagggatgcctgggaatcatttatcatatta

SEQ ID NO: 4 - protein, 416 aa, mus musculus
MASLKRFQTLVPLDHKQGTLFEIIGEPKLPKWFHVECLEDPKRLYVEPRLLEIMFGKDGEHIPHLESMLHTLIHVNVWGPERRAEIWIFGPPPFRRDVDRMLTDLAHYCRMKLMEIEALEAGVERRRMAAHKAATQPAPVKVREAAPQPAPVQEVREAAPQQASVQEEVREAATEQAPVQEVREAATEQAPVQEVSEAATEQAPVQEVNEAATEQASVQAVREAATRPAPGKVRKAATQPAPVQVCQEATQLAPVKVREAATQPASGKVREAATQLAPVKVRKAATQLAPVKVHEAATQPAPGKVSDAATQSASVQVREAATQLSPVEATDTSQLAQVKADEAFAQHTSGEAHQVANGQSPIEVCETATGQHSLDVSRALSQKCPEVFEWETQSCLDGSYVIVQPPRDAWESFIIL

SEQ ID NO: 5 - DNA, 1242 bp, mus musculus
atgggagcaccctcagcatccagatgttctggagtctgcagtaccagtgttccagaaggcttcactcctgagggaagcccggtatttcaggacaaggacatccccgcaattaaccaagggctcatctcagaggagaccccagaaagcagcttcctggtagaaggggacattatccggccaagccctttccgattgttgtcagtgaccaataataaatggcccaagggcgttggtggctttgtggagatccccttcctgctttccagaaagtatgatgaactcagccgccgggtcattatggatgcctttgctgagtttgaacgtttcacatgcatccggtttgttgcctaccatggtcagagagactttgtttccattcttcctatggcggggtgtttctctggtgtgggacgcagtggagggatgcaggtggtgtccttggcacccacttgtctccggaagggccgaggcattgtcctacatgagctcatgcacgtacttggcttctggcatgagcattcacgggcagatcgggaccgctacatccaagtcaactggaacgagatcctcccgggctttgaaatcaacttcatcaagtcacggagtaccaatatgttagttccctatgactactcatctgtgatgcattatgggagatttgccttcagctggcgtgggcagcccaccatcataccactctggacctccagtgttcacattggccagcgatggaacctgagtacctcagatatcacccgggtctgcaggctgtataactgcagccggagtgtccctgactcccacgggagagggtttgaggcccagagtgatggaagcagcctcacccctgcctctatatcacgtctacaaagacttctcgaggcactgtcagaggaatctggaagctctgcccctagtggctccaggactggaggccagagtattgccgggcttggtaacagccagcaaggatgggagcatcctcctcagagcacattcagtgtgggagccttggcaagaccacctcagatgctagccgatgcttcaaaatcggggcctggagcaggtgcagacagcttgtctctagagcagttccagctagcccaggcccccactgtacctcttgctctatttccagaagccagagacaagccagcacctatccaagatgcctttgagaggctagctccacttccaggaggctgtgcacctggaagtcacattagagaggtgcccagagac

SEQ ID NO: 6 - protein, 414 aa, mus musculus
MGAPSASRCSGVCSTSVPEGFTPEGSPVFQDKDIPAINQGLISEETPESSFLVEGDIIRPSPFRLLSVTNNKWPKGVGGFVEIPFLLSRKYDELSRRVIMDAFAEFERFTCIRFVAYHGQRDFVSILPMAGCFSGVGRSGGMQVVSLAPTCLRKGRGIVLHELMHVLGFWHEHSRADRDRYIQVNWNEILPGFEINFIKSRSTNMLVPYDYSSVMHYGRFAFSWRGQPTIIPLWTSSVHIGQRWNLSTSDITRVCRLYNCSRSVPDSHGRGFEAQSDGSSLTPASISRLQRLLEALSEESGSSAPSGSRTGGQSIAGLGNSQQGWEHPPQSTFSVGALARPPQMLADASKSGPGAGADSLSLEQFQLAQAPTVPLALFPEARDKPAPIQDAFERLAPLPGGCAPGSHIREVPRD

SEQ ID NO: 7 - DNA, 1176 bp, mus musculus
atgggagcaccctcagcatccagatgttctggagtctgcagtaccagtgttccagaaggcttcactcctgagggaagcccggtatttcaggacaaggacatccccgcaattaaccaagggctcatctcagaggagaccccagaaagcagcttcctggtagaaggggacattatccggccaggggtcagccacggtgtgtctttcccagatgaactcagccgccgggtcattatggatgcctttgctgagtttgaacgtttcacatgcatccggtttgttgcctaccatggtcagagagactttgtttccattcttcctatggcggggtgtttctctggtgtgggacgcagtggagggatgcaggtggtgtccttggcacccacttgtctccggaagggccgaggcattgtcctacatgagctcatgcacgtacttggcttctggcatgagcattcacgggcagatcgggaccgctacatccaagtcaactggaacgagatcctcccgggctttgaaatcaacttcatcaagtcacggagtaccaatatgttagttccctatgactactcatctgtgatgcattatgggagatttgccttcagctggcgtgggcagcccaccatcataccactctggacctccagtgttcacattggccagcgatggaacctgagtacctcagatatcacccgggtctgcaggctgtataactgcagccggagtgtccctgactcccacgggagagggtttgaggcccagagtgatggaagcagcctcacccctgcctctatatcacgtctacaaagacttctcgaggcactgtcagaggaatctggaagctctgcccctagtggctccaggactggaggccagagtattgccgggcttggtaacagccagcaaggatgggagcatcctcctcagagcacattcagtgtgggagccttggcaagaccacctcagatgctagccgatgcttcaaaatcggggcctggagcaggtgcagacagcttgtctctagagcagttccagctagcccaggcccccactgtacctcttgctctatttccagaagccagagacaagccagcacctatccaagatgcctttgagaggctagctccacttccaggaggctgtgcacctggaagtcacattagagaggtgcccagagac

SEQ ID NO: 8 - protein, 392 aa, mus musculus
MGAPSASRCSGVCSTSVPEGFTPEGSPVFQDKDIPAINQGLISEETPESSFLVEGDIIRPGVSHGVSFPDELSRRVIMDAFAEFERFTCIRFVAYHGQRDFVSILPMAGCFSGVGRSGGMQVVSLAPTCLRKGRGIVLHELMHVLGFWHEHSRADRDRYIQVNWNEILPGFEINFIKSRSTNMLVPYDYSSVMHYGRFAFSWRGQPTIIPLWTSSVHIGQRWNLSTSDITRVCRLYNCSRSVPDSHGRGFEAQSDGSSLTPASISRLQRLLEALSEESGSSAPSGSRTGGQSIAGLGNSQQGWEHPPQSTFSVGALARPPQMLADASKPGPGAGADSLSLEQFQLAQAPTVPLALFPEARDKPAPIQDAFERLAPLPGGCAPGSHIREVPRD

SEQ ID NO: 9 - DNA, 1140 bp, mus musculus
atgggagcaccctcagcatccagatgttctggagtctgcagtaccagtgttccagaaggcttcactcctgagggaagcccggtatttcaggacaaggacatccccgcaattaaccaagggctcatctcagaggagaccccagaaagcagcttcctgctttccagaaagtatgatgaactcagccgccgggtcattatggatgcctttgctgagtttgaacgtttcacatgcatccggtttgttgcctaccatggtcagagagactttgtttccattcttcctatggcggggtgtttctctggtgtgggacgcagtggagggatgcaggtggtgtccttggcacccacttgtctccggaagggccgaggcattgtcctacatgagctcatgcacgtacttggcttctggcatgagcattcacgggcagatcgggaccgctacatccaagtcaactggaacgagatcctcccgggctttgaaatcaacttcatcaagtcacggagtaccaatatgttagttccctatgactactcatctgtgatgcattatgggagatttgccttcagctggcgtgggcagcccaccatcataccactctggacctccagtgttcacattggccagcgatggaacctgagtacctcagatatcacccgggtctgcaggctgtataactgcagccggagtgtccctgactcccacgggagagggtttgaggcccagagtgatggaagcagcctcacccctgcctctatatcacgtctacaaagacttctcgaggcactgtcagaggaatctggaagctctgcccctagtggctccaggactggaggccagagtattgccgggcttggtaacagccagcaaggatgggagcatcctcctcagagcacattcagtgtgggagccttggcaagaccacctcagatgctagccgatgcttcaaaatcggggcctggagcaggtgcagacagcttgtctctagagcagttccagctagcccaggcccccactgtacctcttgctctatttccagaagccagagacaagccagcacctatccaagatgcctttgagaggctagctccacttccaggaggctgtgcacctggaagtcacattagagaggtgcccagagac

SEQ ID NO: 10 - protein, 380 aa, Mus musculus
MGAPSASRCSGVCSTSVPEGFTPEGSPVFQDKDIPAINQGLISEETPESSFLLSRKYDELSRRVIMDAFAEFERFTCIRFVAYHGQRDFVSILPMAGCFSGVGRSGGMQVVSLAPTCLRKGRGIVLHELMHVLGFWHEHSRADRDRYIQVNWNEILPGFEINFIKSRSTNMLVPYDYSSVMHYGRFAFSWRGQPTIIPLWTSSVHIGQRWNLSTSDITRVCRLYNCSRSVPDSHGRGFEAQSDGSSLTPASISRLQRLLEALSEESGSSAPSGSRTGGQSIAGLGNSQQGWEHPPQSTFSVGALARPPQMLADASKSGPGAGADSLSLEQFQLAQAPTVPLALFPEARDKPAPIQDAFERLAPLPGGCAPGSHIREVPRD

SEQ ID NO: 11 - DNA, 384 bp, mus musculus
aaggtcttcagtcgctgtgagctggccaaagagatgcatgacttcggtctggatggctaccggggttataacctggctgactgggtctgccttgcttactacacaagtggcttcaacacaaatgctgtggatcatgaagctgatggaagcaccaacaatggcatcttccagatcagcagccggaggtggtgcagaaccctcgcctcgaatggccccaatctttgcaggatatactgcactgatttgttgaacaatgatctcaaagattctatcgtctgtgccatgaagatagttcaagaacccctgggtctgggctattgggaagcctggaggcaccactgccagggcagggacctcagtgactgggtggatggctgtgacttc

SEQ ID NO: 12 - protein, 128 aa, mus musculus
KVFSRCELAKEMHDFGLDGYRGYNLADWVCLAYYTSGFNTNAVDHEADGSTNNGIFQISSRRWCRTLASNGPNLCRIYCTDLLNNDLKDSIVCAMKIVQEPLGLGYWEAWRHHCQGRDLSDWVDGCDF

SEQ ID NO: 13 - DNA, 820 bp, mus musculus
gccctggcaaggttgtgggggacatcttgagctgaagcagggttttgagccactgctgctgctgccattgtcaccatggtctcagctctgcggggagcacccctgatcagggtgcactcaagccctgtttcttctccttctgtgagtggaccacggaggctggtgagctgcctgtcatcccaaagctcagctctgagccagagtggtggtggctccacctctgccgccggcatagaagccaggagcagggctctcagaaggcggtggtgcccagctgggatcatgttgttggccctggtctgtctgctcagctgcctgctaccctccagtgaggccaagctctacggtcgttgtgaactggccagagtgctacatgacttcgggctggacggataccggggatacagcctggctgactgggtctgccttgcttatttcacaagcggtttcaacgcagctgctttggactacgaggctgatgggagcaccaacaacgggatcttccagatcaacagccggaggtggtgcagcaacctcaccccgaacgtccccaacgtgtgccggatgtactgctcagatttgttgaatcctaatctcaaggataccgttatctgtgccatgaagataacccaagagcctcagggtctgggttactgggaggcctggaggcatcactgccagggaaaagacctcactgaatgggtggatggctgtgacttctaggatggacggaaccatgcacagcaggctgggaaatgtggtttggttcctgacctaggcttgggaagacaagccagcgaataaaggatggttgaacgtt

SEQ ID NO: 14 - protein, 215 aa, homo sapien [sequence omitted]

SEQ ID NO: 15 - DNA, 417 bp, mus musculus
aagatttatgaacgctgtgagctggcaaagaagctggaggaggctggcctcgatggcttcaaaggctatactgttggagactggctgtgtgtggcacactatgagagtggctttgacacctcttttgtggaccacaatccagatggcagcagtgaatatggcattttccagctgaactctgcctggtggtgtaacaatggcatcacacccactcagaacctctgcaacatcgattgtaatgacctgctcaaccgccatattctggatgatatcatatgtgccaagagggttgcatcctcacataagagtatgaaggcctgggattcctggacccagcactgtgccggtcatgatttatcagaatggctaaaggggtgttctgtgcgtctgaaaactgactcaagctataataactgg

SEQ ID NO: 16 - protein, 139 aa, mus musculus
KIYERCELAKKLEEAGLDGFKGYTVGDWLCVAHYESGFDTSFVDHNPDGSSEYGIFQLNSAWWCNNGITPTQNLCNIDCNDLLNRHILDDIICAKRVASSHKSMKAWDSWTQHCAGHDLSEWLKGCSVRLKTDSSYNNW

SEQ ID NO: 17 - DNA, 588 bp, homo sapien
ctgggagggcttacaggtgccataatgaaggcctggggcactgtggtagtgaccttggccacgctgatggttgtcactgtggatgccaagatctatgaacgctgcgagctggcggcaagactggagagagcagggctgaacggctacaagggctacggcgttggagactggctgtgcatggctcattatgagagtggctttgacaccgccttcgtggaccacaatcctgatggcagcagtgaatatggcattttccaactgaattctgcctggtggtgtgacaatggcattacacccaccaagaacctctgccacatggattgtcatgacctgctcaatcgccatattctggatgacatcaggtgtgccaagcagattgtgtcctcacagaatgggctttctgcctggacttcttggaggctacactgttctggccatgatttatctgaatggctcaaggggtgtgatatgcatgtgaaaattgatccaaaaattcatccatgactcagattcgaagagacagattttatcttcctttcatttctttctcttgtgcatttaataaaggatggtatctataaacaatgc

SEQ ID NO: 18 - protein, 159 aa, homo sapien
MKAWGTVVVTLATLMVVTVDAKIYERCELAARLERAGLNGYKGYGVGDWLCMAHYESGFDTAFVDHNPDGSSEYGIFQLNSAWWCDNGITPTKNLCHMDCHDLLNRHILDDIRCAKQIVSSQNGLSAWTSWRLHCSGHDLSEWLKGCDMHVKIDPKIHP